(19) United States
(12) Patent Application Publication
(10) Pub. No.: US 2025/0312678 A1
(43) Pub. Date: Oct. 9, 2025

(54) EXTENDED REALITY BASED NEUROMOTOR REHABILITATION

(71) Applicant: Neuro Group XR Inc., Montreal (CA)

(72) Inventors: Nicolas Jimenez, Santiago (CL); Leandro Palmieri, Madrid (ES); Benjamin Peralta, Madrid (ES)

(73) Assignee: Neuro Group XR Inc., Montreal (CA)

(21) Appl. No.: 19/169,087

(22) Filed: Apr. 3, 2025

Related U.S. Application Data
(60) Provisional application No. 63/631,671, filed on Apr. 9, 2024.

(51) Int. Cl. A63B 71/06; A63B 24/00; G16H 20/30; G16H 50/50

(52) U.S. Cl. CPC A63B 71/0622; A63B 24/0003; G16H 20/30; G16H 50/50; A63B 2024/0065; A63B 2071/0636; A63B 2220/20

(57) ABSTRACT

A system can include one or more processors, coupled with memory, to receive, from extended reality equipment, a sensed movements of a hand of a patient from the extended reality equipment. The system can animate the hand of the patient on the extended reality equipment based on the sensed movements.

### CROSS-REFERENCE TO RELATED PATENT APPLICATION

This application claims the benefit of, and priority to, U.S. Provisional Patent Application No. 63/631,671 filed Apr. 9, 2024, the entirety of which is incorporated by reference herein.

### BACKGROUND

A patient can have a neuromotor impairment. The neuromotor impairment can be the result of a condition or injury. The neuromotor impairment can affect impulses between the brain, spinal cord, and nervous system of a patient and muscles of the patient, e.g., hand muscles, finger muscles, or arm muscles. The neuromotor impairment can reduce a patient's neuromotor control of their hands, fingers, arms, or head. A patient with a neuromotor impairment can have difficulty moving their arms, hands, head, or fingers to grasp objects with their hands, move the objects with their hands, or perform everyday tasks.

### SUMMARY

At least one aspect of the present disclosure is directed to a system. The system can include one or more processors, coupled with memory. The one or more processors can receive, from extended reality equipment, a sensed movement of a hand of a patient attempting to grasp a virtual object displayed on the extended reality equipment. The one or more processors can select a level of assistance to provide the patient to grasp the virtual object using a level of rehabilitation of the patient. The one or more processors can animate, on the extended reality equipment, the hand of the patient grasping the virtual object using the level of assistance.

At least one aspect of the present disclosure is directed to a method. The method can include receiving, by one or more processors, from extended reality equipment, a sensed movement of a hand of a patient attempting to grasp a virtual object displayed on the extended reality equipment. The method can include selecting, by the one or more processors, a level of assistance to provide the patient to grasp the virtual object using a level of rehabilitation of the patient. The method can include animating, by the one or more processors, on the extended reality equipment, the hand of the patient grasping the virtual object using the level of assistance.

At least one aspect of the present disclosure is directed to one or more computer readable media storing instructions thereon, that, when executed by one or more processors, cause the one or more processors to perform operations. The operations can include receiving, from extended reality equipment, a sensed movement of a hand of a patient attempting to grasp a virtual object displayed on the extended reality equipment. The operations can include selecting a level of assistance to provide the patient to grasp the virtual object using a level of rehabilitation of the patient. The operations can include animating, on the extended reality equipment, the hand of the patient grasping the virtual object using the level of assistance.

At least one aspect of the present disclosure is directed to a system. The system can include one or more processors, coupled with memory. The one or more processors can animate, on extended reality equipment, a computer rendered environment including a virtual object and a first target for a patient to move the virtual object from a starting position along a first axis to the first target. The one or more processors can receive, from the extended reality equipment, sensed movements of a hand of the patient grasping the virtual object and moving the virtual object along the first axis to the first target. The one or more processors can update, on the extended reality equipment, the computer rendered environment to include a second target for the patient to move the virtual object along a second axis to responsive to the sensed movements of the hand of the patient indicating at least a threshold level of rehabilitation.

At least one aspect of the present disclosure is directed to a method. The method can include animating, by one or more processors, on extended reality equipment, a computer rendered environment including a virtual object and a first target for a patient to move the virtual object from a starting position along a first axis to the first target. The method can include receiving, by the one or more processors, from the extended reality equipment, sensed movements of a hand of the patient grasping the virtual object and moving the virtual object along the first axis to the first target. The method can include updating, by the one or more processors, on the extended reality equipment, the computer rendered environment to include a second target for the patient to move the virtual object along a second axis to responsive to the sensed movements of the hand of the patient indicating at least a threshold level of rehabilitation.

At least one aspect of the present disclosure is directed to one or more computer readable media storing instructions thereon, that, when executed by one or more processors, cause the one or more processors to perform operations. The operations can include animating, on extended reality equipment, a computer rendered environment including a virtual object and a first target for a patient to move the virtual object from a starting position along a first axis to the first target. The operations can include receiving, from the extended reality equipment, sensed movements of a hand of the patient grasping the virtual object and moving the virtual object along the first axis to the first target. The operations can include updating, on the extended reality equipment, the computer rendered environment to include a second target for the patient to move the virtual object along a second axis to responsive to the sensed movements of the hand of the patient indicating at least a threshold level of rehabilitation.

At least one aspect of the present disclosure is directed to a system. The system can include one or more processors, coupled with memory. The one or more processors can receive, from extended reality equipment, sensed movements of a portion of a patient attempting to move a virtual object in a computer rendered environment displayed on the extended reality equipment. The one or more processors can generate, using the sensed movements, a three-dimensional frequency heat map indicating movements of the portion of the patient in the computer rendered environment. The one or more processors can execute a model trained by machine learning using the three-dimensional frequency heat map to determine a level of rehabilitation of the patient.

At least one aspect of the present disclosure is directed to a method. The method can include receiving, by one or more processors, from extended reality equipment, sensed movements of a portion of a patient attempting to move a virtual object in a computer rendered environment displayed on the extended reality equipment. The method can include generating, by the one or more processors, using the sensed movements, a three-dimensional frequency heat map indicating movements of the portion of the patient in the computer rendered environment. The method can include executing, by the one or more processors, a model trained by machine learning using the three-dimensional frequency heat map to determine a level of rehabilitation of the patient.

At least one aspect of the present disclosure is directed to one or more computer readable media storing instructions thereon, that, when executed by one or more processors, cause the one or more processors to perform operations. The operations can include receiving from extended reality equipment, sensed movements of a portion of a patient attempting to move a virtual object in a computer rendered environment displayed on the extended reality equipment. The operations can include generating using the sensed movements, a three-dimensional frequency heat map indicating movements of the portion of the patient in the computer rendered environment. The operations can include executing a model trained by machine learning using the three-dimensional frequency heat map to determine a level of rehabilitation of the patient.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example computing system for extended reality (XR) based neuromotor rehabilitation.

FIGS. 2-3 depict example layouts of a XR environment for a patient to move a virtual object along a horizontal axis to a target.

FIGS. 4-5 depict example layouts of a XR environment for a patient to move a virtual object along a vertical axis to a target.

FIGS. 6-7 depict example layouts of a XR environment for a patient to move virtual objects along a vertical axis and a horizontal axis to targets.

FIGS. 8-9 depict example layouts of a XR environment for a patient to move virtual objects spaced in a semi-circle along axes to targets.

FIG. 10 depicts an example XR environment for a patient to move virtual objects spaced in a semi-circle along axes to targets.

FIG. 11 depicts an electronic display including two-dimensional heat maps depicting performance of a patient moving virtual objects along a horizontal axis to a target.

FIG. 12 depicts an electronic display including two-dimensional heat maps depicting performance of a patient moving virtual objects in a direction along a vertical axis to targets.

FIG. 13 depicts an electronic display including two-dimensional heat maps depicting positions of a hand and a head of a patient.

FIG. 14A depicts a XR environment tracking a three-dimensional heat map depicting positions of a hand of a patient.

FIG. 14B depicts a MR environment tracking a three-dimensional heat map depicting positions of a hand of a patient.

FIG. 14C depicts a MR environment tracking a three-dimensional heat map anchored in a MR environment depicting positions of a hand of a patient.

FIG. 15 depicts a XR environment including performance indicators of hand poses.

FIG. 16 depicts an example method of selecting a level of assistance to provide a patient to grasp a virtual object.

FIG. 17 depicts an example method of updating a XR environment based on a level of rehabilitation of a patient.

FIG. 18 depicts an example method of generating a heat map depicting positions of a hand of a patient in a XR environment.

FIG. 19 depicts an example computing architecture.

### DETAILED DESCRIPTION

Following below are more detailed descriptions of various concepts related to, and implementations of, methods, apparatuses, and systems for mixed reality (XR) (e.g., virtual reality (VR), augmented reality (AR), or mixed reality (MR)) based neuromotor rehabilitation. The various concepts introduced above and discussed in greater detail below may be implemented in any of numerous ways.

A first aspect of this disclosure is generally directed to tracking a hand of a patient in a XR environment. It may be difficult for an extended reality computing system to provide realistic and accurate experiences for tracking and animating hands and fingers of a patient in extended reality. For example, extended reality systems may provide unrealistic animated representations of the fingers and hands of a patient because the representations of fingers or hands of a patient may not accurately conform to the actual physical positions of the fingers or hands of the patient. This reduces functional integration and immersion in a XR environment. The accuracy limitations between animated fingers and hands of a patient and the actual positions of the fingers and hands of the patient reduces the usefulness of extended reality applications, specifically for neuromotor rehabilitation therapy. This lack of realism and accuracy in hand tracking hinders neuromotor rehabilitation therapy, because accurate animation of the fingers or hands of a patient can be important to assess and improve patient neuromotor functions.

Furthermore, hand tracking in extended reality may not be personalized in order to rehabilitate patients of various ability levels. For example, even if a extended reality system can accurately track movements of a hand of a patient and animate a virtual representation of the hands of the patient, the extended reality system tracking may not be useful for a patient with a low level of neuromotor ability. Therefore, the extended reality system tracking may lack the flexibility to adapt to the needs of patients.

To solve these, and other technical problems, technical solutions of this disclosure can include an extended reality computing system that switches between levels of assistance to provide a patient in animating the hand of the patient. The extended reality computing system can allow a personalized and tailored level of interaction to meet the specific functional needs of patients with different degrees of disability. The extended reality computing system can switch between providing various levels of assistance to the patient by automating movement of fingers or hands of a patient in an extended reality environment, depending on determined abilities of the patients. For example, for an advanced patient, the extended reality computing system can animate the hand of finger of a patient to closely correspond to the actual position of a hand or position of a finger. For a less advanced patient, the computing system can animate the movement hand or finger of a patient to a position where the patient is attempting to move their hand or finger, although the patient is unable to actually move their physical hand or finger to the desired position due to neuromotor disability.

The extended reality computing system can provide highly accurate hand tracking, and the animated representation of a hand or finger of a patient can closely match the actual hand or finger of the patient. The computing system can integrate multiple assets (e.g., software development kits (SDKs, development tools, applications, graphics components) together to enable the system to provide accurate hand and finger tracking. For example, the system can integrate an extended reality asset with a collider asset. The collider asset can animate collisions or interactions between a hand or finger of a patient with a virtual object. The collider asset can provide specialized hand physics tracking and gesture recognition that enables various levels of grips or gripping postures of a virtual object. The extended reality asset can provide computer vision and artificial intelligence functions for hand tracking. The extended reality asset can configure object physics. The extended reality computing system can run at least one script to integrate the assets together to coordinate the assets and provide accurate tracking and representation of a hand or finger in a XR environment, and the interactions of the hands or fingers of the patient with virtual objects. By combining the collider asset and the extended reality asset, the computing system can implement accurate hand tracking with natural interaction, gradable and adaptable to virtual objects and environments. The extended reality computing system can use the collider asset and the extended reality asset together to provide a realistic representation of hands, fingers, and object physics in the XR environment. This combination of assets and improved hand representation can improve rehabilitation therapies in a extended reality environment.

A second aspect of this disclosure is generally directed to design of extended reality based neuromotor rehabilitation therapy. A system that uses extended reality to implement neuromotor rehabilitation therapy can have challenges, such as adapting to users with various levels and types of motor and cognitive impairments. The computing system can implement neuromotor rehabilitation that may not take into account the needs of the patient, medical and clinical research findings, or clinical experiences of rehabilitators and therapists. The absence of a methodology that considers users with motor-cognitive impairments of varying severity in the design of the software, that does not incorporate evidence-based principles of motor learning in its practice, and that does not offer experiences adapted to different levels of human functioning, can hinder and limit effective neuromotor rehabilitation.

To solve these, and other technical problems, technical solutions of this disclosure can include an extended reality computing system that implements extended reality rehabilitation that adapts to the needs of a patient. The system can identify and track the rehabilitation of a patient, and increase the difficulty of the tasks to be completed by a patient as a patient advances in their rehabilitation. The extended reality computing system can combine neurorehabilitation training experiences, clinical resource that subscribes to the conceptual framework of the International Classification of Functioning (ICF), the functional needs related to the taxonomy of upper extremity movement and evidence-based motor learning principles, functional movement taxonomy, and principles of motor learning. The extended reality computing system can combine these clinical-conceptual frameworks to include patients with motor and cognitive impairments in extended reality experiences in the three dimensions of human functioning, the computing system can provide the practice of functional movements appropriate to their individual abilities and needs and favor motor recovery with specific trainings that integrate principles of motor learning and neuroplasticity.

The computing system can animate a XR environment including a target for a user to move a virtual object along an axis to a target. The computing system can track the performance of the patient in moving the virtual object to the target, and update the target over time. For example, as the patient improves their motor skills, the computing system can change the axis for the user to move the virtual objects along, animate XR environments of the user to move virtual objects along multiple axis at once, have a semi-circle of axes, etc. The computing system can update and animate the XR environment to correspond to the rehabilitation progression of the patient.

A third aspect of this disclosure is generally directed to automating rehabilitation in extended reality with metrics. A extended reality computing system may not efficiently visualize and analyze the progression of a patient's neuromotor rehabilitation. A extended reality computing system may have limitations in the collection of functional metrics and in the presentation of accurate performance-related data associated with the distance between the fingers of the hand, the work area or hand movement in three dimensions, and the positioning and translation of the head. This lack of accuracy in the metrics collected can prevent the extended reality computing system from performing a detailed assessment and effective visualization of the progress of a patient. This can hinder the adaptation and progression of neuromotor rehabilitation of a patient.

Furthermore, the extended reality system may not be able to accurately personalize the rehabilitation training of a patient. A therapist or other user may attempt to personalize the extended reality-based training of the patient, but the personalization may be subjective and inaccurate. For a therapist to tailor rehabilitation to a patient, the therapist may need to attempt to manually tailor the rehabilitation to the patient with extensive customizations, which may still be inaccurate. These shortcomings underline the need for a solution that comprehensively addresses therapeutic interactions with clear visualization of outcomes and adaptation to patient's rehabilitation progress, such as daily progress.

To solve these, and other technical problems, technical solutions of this disclosure can include collecting metrics that track the rehabilitation of a patient, and using machine learning to personalize the rehabilitation to individual patients. An extended reality computing system can accurately collect metrics, such as the distance between the fingers of the hand, various functional postures of the hand, a motion or position heatmap or frequency map of the hand in space (e.g., a two-dimensional or three-dimensional heatmap), or a motion or position heatmap or frequency map of a head of the patient in a work area.

The extended reality computing system can train a model with machine learning to identify a type of rehabilitation to provide a user based on the collected metrics and heatmaps. For example, the model can be trained with other metrics or other heatmaps to identify the performance or level of rehabilitation of a patient. The model can, for a given set of metrics or heatmaps, output a level of rehabilitation of a patient that a therapist could not otherwise precisely identify. Using machine learning, the extended reality computing system can automate exercise adaptation with proper progression and customization. The exercises can be adapted to follow efficient protocols with detailed daily assessments and effective visualization of patient progression. This can provide more efficient rehabilitation tailored to the individual needs of each patient. The extended reality computing system, according to initial and specific capabilities of patients, can challenge a patient in a progressive manner tailored to each patient to achieve recovery, which can overcome inefficiency of other systems, which often rely on extensive manual interventions and extensive customizations. This unique combination of immersive technologies, accurate metrics tracking, and machine learning can enable detailed three-dimensional performance visualization and progressive adaptation of the rehabilitation program, accelerating recovery time with functional performance reports that support the recovery process from hospitals and homes. This can provide more effective outcomes, with feedback to both the patient and the practitioner, delivering a higher quality of care.

The computing system can accurately collect of metrics such as finger spacing, hand and head movement frequency maps, and integrate machine learning to automate, adapt, and customize rehabilitation exercises. These features provide several significant advantages. For example, the computing system can provide accurate metric collection of detailed metrics, enabling accurate daily assessment and effective visualization of patient progression and functional performance. The computing system can implement an immersive, personalized, and efficient rehabilitation experience. The integration of machine learning can allow for automation and continuous adaptation of rehabilitation exercises according to the specific needs of each patient, offering a more efficient and adapted rehabilitation. The computing system can include a detailed progression visualization to visualize functional reports in three dimensions in real-time with relevant details of the progression and daily performance of patients. The computing system can provide tools for informed decision making and proper progression of rehabilitative treatments. The computing system can provide an immersive and motivating experience, the solution can offer a real-life based immersive experience that directly engages affective-emotional systems that directly increases motivation to patients, overcoming the lack of engagement commonly experienced in conventional therapies. The computing system can provide efficiency and adaptability. The adaptability of hand tracking and interactions with virtual objects according to the capabilities of each patient allows them to obtain satisfactory experiences with little or no movement, deceiving the brain and opening the possibilities of transferring what has been achieved in the virtual world to the real world in a faster and easier way.

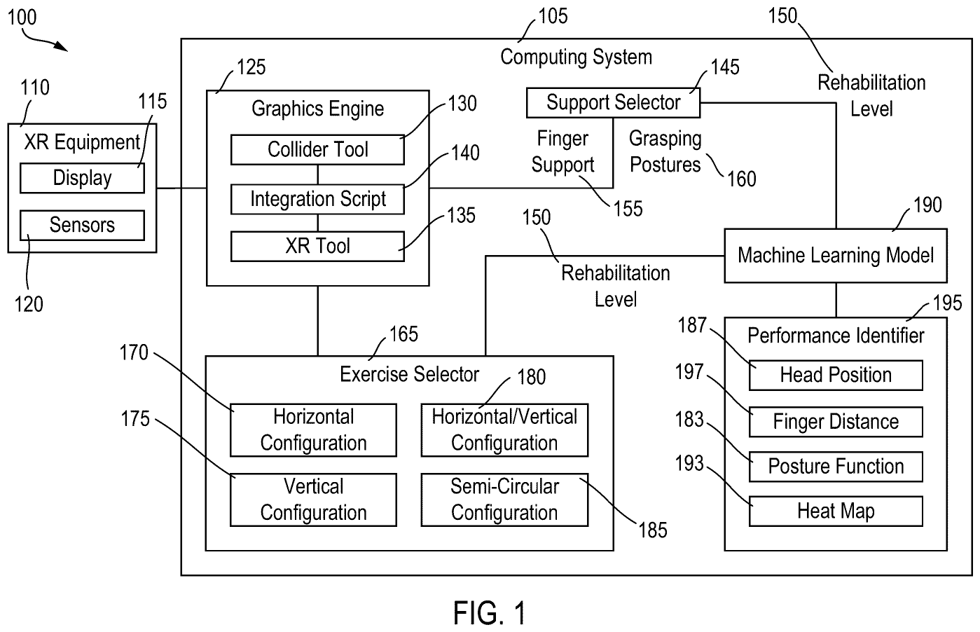

Referring now to FIG. 1, among others, a system 100 including a computing system 105 for extended reality based neuromotor rehabilitation is shown. The computing system 105 can be an extended reality system to provide motor and cognitive patient rehabilitation in XR (e.g., VR, MR, and/or AR). The patient can be an adult, a teenager, or a child with a neuromotor impairment. The system 100 can include extended reality (XR) equipment 110, such as augmented reality equipment, VR equipment, or MR equipment. The XR equipment 110 can be or include wearable devices. For example, the XR equipment 110 can include a headset, goggles, glasses, gloves, finger sensors, etc. that are worn on the head, arms, hands, or fingers of a patient. The XR equipment 110 can include at least one display 115. The XR equipment 110 can include a display 115 for a right eye of a patient and a display 115 for a left eye of a patient. The display 115 can be an organic light emitting diode display (OLED), a liquid crystal display (LCD), or a light emitting diode (LED) display worn on a head of a patient. The XR equipment 110 can cause the display 115 to display images of a virtual environment or world or images of virtual objects or structures. The XR equipment 110 can display fully immersive VR, or can overlay information over a real-world view of a patient (e.g., either directly seen by the patient or captured via cameras and then displayed to the patient).

The XR equipment 110 can include at least one sensor 120. The sensor 120 can be a motion sensor, such as an inertial measurement unit (IMU). The IMU can be or include a gyroscope, an accelerometer, or a magnetometer. The IMU can sense accelerations along three body axes (x, y, and z-axes of the IMU) and rotational accelerations about each body axes. The sensor 120 can be coupled with or worn by an appendage of a patient to detect or sense the position, orientation, rotation, or movement of an appendage of the patient. For example, the sensor 120 can track the motions or movements of a left arm or a right arm of a patient. The sensor 120 can track the position or rotation of a torso of a patient. The sensor 120 can track the position or rotation of a head or neck of a patient. The sensor 120 can track the position or movement of a hand of a patient, such as a left hand or right hand. The sensor 120 can track the position or movement of a finger of a patient. The sensor 120 can track the position or movement of individual fingers of a patient, e.g., a thumb, a little finger, a ring finger, a middle finger, an index finger. The sensors 120 can be wearable sensors that are portable and worn by a patient. The sensors 120 can be external or global capture sensors.

The system 100 can include at least one computing system 105, such as an extended reality computing system. At least a portion of the computing system 105 can part of, or integrated with the XR equipment 110. At least a portion of the computing system 105 can be separate from the extended reality system 100. The computing system 105 can be a mobile computing system, such as a smartphone, tablet, laptop, computer, or desktop computer. The computing system 105 can be a server system, a remote-computing system, or a cloud computing system. The computing system 105 can be a data processing system, microprocessor system, or embedded system.

The computing system 105 can include at least one graphics engine 125. The graphics engine 125 can be a graphics engine such as UNREAL ENGINE, UNITY, CRYENGINE, etc. The graphics engine 125 can generate, render, animate, draw, or produce an XR environment, e.g., a VR environment, a MR environment, or AR environment for the equipment 110. The XR environment can be a fully or partially computer generated environment or computer rendered environment. The graphics engine 125 can animate the position, movement, color, or appearance of virtual elements, such as a hand of a patient, an object, a graphic, furniture, a table, etc. For example, the computing system 105 can execute the graphics engine 125 to animate, on the XR equipment 110, the hand of the patient grasping the virtual object. For example, the graphics engine 125 can animate the hands or arms of the patient moving according to sensed movements received from the sensors 120. The graphics engine 125 can animate the hands of the patient grasping, picking, dropping, or setting down virtual objects according to the sensed movements of the fingers or hands of the patients via the sensors 120.

The graphics engine 125 can include at least one collider tool 130. The collider tool 130 can be a first tool of multiple tools run, executed, or operated by the graphics engine 125. For example, the collider tool 130 can be a tool, asset, software development kit, software development tool, etc., such as asset HPTK. The collider tool 130 can provide specialized hand physics tracking and gesture recognition that enables a grip with multiple levels of support when grasping any virtual object. The collider tool 130 can model physics to animate collisions between a hand of the patient and a virtual object. The collider tool 130 can provide hand physics tracking and gesture recognition based on sensed motions or movements of the hands or fingers of the patient captured via the sensors 120. The collider tool 130 can provide logic and physics modeling to animate various hand poses and snap hands to virtual objects according to sensed motion data received from the sensors 120. Snapping a hand of a patient to a virtual object can lock or fix a virtual representation of a hand of the patient to the virtual object.

The graphics engine 125 can include at least one XR tool 135. The XR tool 135 can be a second tool of multiple tools run, executed, or operated by the graphics engine 125. For example, the XR tool 135 can be a tool, asset, software development kit, software development tool, etc., such as OCULUS INTEGRATION. The XR tool 135 can provide extended reality functions for tracking and animating the arms, hands, or fingers of a patient. For example, the XR tool 135 can implement computer vision and artificial intelligence to track or animate the movements of the arms, hands, or fingers of a patient. The XR tool 135 can configure the three- dimensional objects. For example, the XR tool 135 can configure physics for the virtual objects to animate their movements, motions, etc. The XR tool 135 can optimize virtual interactions in an intuitive, functional, and realistic way.

The graphics engine 125 can integrate the collider tool 130 with the XR tool 135. For example, the graphics engine 125 can execute at least one integration script 140 to integrate the collider tool 130 with the XR tool 135. The graphics engine 125 can execute one, or a set of scripts 140 that integrate the collider tool 130 with the XR tool 135. The scripts 140 can include multiple operations that integrate functions of the collider tool 130 and functions of the XR tool 135. The script 140 can be a component or function of an avatar that represents the position or orientation of a patient. For example, the script can be a part or function of an object that defines the avatar. The object can define the avatar's animations, graphic models, or animation rules. The object can connect the sensed movements received from the sensors 120 to animations of the avatar in order to replicate or mirror the postures, positions, or movements of the patient. The script 140 can be executed by the graphics engine 125 along with execution of the instructions of the avatar.

The script 140 can integrate the computing system 105 or the graphics engine 125 with the collider tool 130 and the XR tool 135. The script 140 can act as an intelligent intermediary that recognizes and manages avatar interaction levels, adapting to the varying degrees of assistance during the extended reality experience. For example, the script 140 can implement the various levels of finger support 155 and grasping postures 160. The script 140 can dynamically identify the appropriate development kit or tool (e.g., the collider tool 130 or XR tool 135) to be employed or executed by the graphics engine 125 based on the specific needs of the patient, ensuring seamless integration and a consistent user experience. The integration script 140 can provide adaptability and enable a seamless transition between different development environments, such as the collider tool 130 and the XR tool 135, optimizing interactivity and user immersion the XR environment.

For example, the script 140 can identify whether to execute the collider tool 130 or the XR tool 135. The script 140 can cause the graphics engine 125 to execute the identified tool. For example, the script 140 can receive the selected finger support level 155 or the grasping posture level 160. In some implementations the script 140 can implement the operations of the support selector 145 to determine the finger support level 155 or the grasping postures 160 from the rehabilitation level 150 itself. For example, if the integration script 140 identifies that realistic grasping should be implemented (e.g., a low level of finger support 155), the script 140 can activate the collider tool 130 and cause the collider tool 130 to run to animate the movement of fingers or hands of a patient and collisions or interactions between the fingers or hands of the patient and the virtual objects. If the script 140 identifies that medium or high level of finger support 155 should be implemented, the script 140 can cause the XR tool 135 to run to provide the medium or high level of finger support 155 and animate the movements of the hands or fingers of the patient and the collisions or interactions between the fingers or hands of the patient and the virtual objects. Likewise, the script 140 can cause the collider tool 130 to run to implement a high or medium number of grasping postures 160 and run the XR tool 135 to implement a medium or low number of grasping postures 160. The script 140 can deactivate or stop a tool 130 or 135 from running if the script 140 identifies that the particular tool 130 or 135 is not used to implement the selected level of finger support 155 or the selected grasping postures 160. By integrating the collider tool 130 and the XR tool 135 together, the graphics engine 125 can significantly improve the accuracy and quality of hand representation in VR, offering a realistic and highly tailored experience for a patient.

The computing system 105 can include at least one support selector 145. The support selector 145 can select a level of assistance to provide a user during training or therapy. For example, for a patient with a neuromotor disability, the patient may not be able to fully close their fingers to grasp a virtual object. The patient may close their fingers to attempt to move the fingers to positions to hold the virtual object, but their neuromotor disability may prevent them from doing this. The graphics engine 125 can assist the patient by animating the fingers moving to the intended locations to hold the virtual object, even if the sensed motions via the sensors 120 do not indicate that the patient has actually moved their fingers to those locations. For example, if the graphics engine 125 detects, via the sensors 120, that the patient moved their fingers at least a distance to the intended locations to hold the virtual object, e.g., at least a percentage of a distance between an open hand position and the surface of the virtual object (e.g., 20%, 40%, 60%, etc.), the graphics engine 125 can animate the fingers moving the remaining distance.

The support selector 145 can receive, from the XR equipment 110 sensed movements of a hand of a patient attempting to grasp a virtual object displayed on the XR equipment 110. For example, the support selector 145 can receive the sensed movements of the hand or fingers of the patient via the sensors 120. Based on a level of rehabilitation 150 of the patient, the support selector 145 can select a level of assistance to provide the patient. The support selector 145 can receive a rehabilitation level 150, and update the level of assistance to provide the patient based on changes in the rehabilitation level 150. The levels of assistance provided to the patient can be implemented through the integration between the collider tool 130 and the XR tool 135.

For a new patient, the graphics engine 125 can provide a patient with an initial benchmarking phase where the patient picks up and moves virtual objects, makes different hand posture, etc. The computing system 105 can generate a rehabilitation level 150 for the new patient to accurately select the level of support or the correct exercise layout configuration to provide the patient based on their neuromotor capabilities.

The selected level of assistance can assist the patient to grasp virtual objects. The assistance can be finger support 155. The support selector 145 can store multiple different finger support levels. The support selector 145 can provide any number of different finger support levels, e.g., two, three, four, or more. The finger support levels 155 can be adjusted to the level of functionality or disability of each patient.

For example, each (or some) finger support levels 155 can define a distance that the actual finger of the patient must move towards an intended position before the graphics engine 125 animates the finger of the patient moving or bending to the intended position. The finger support levels 155 can include a first level. When executing with the first finger support level 155, the graphics engine 125 can animate the hand grasping the virtual object responsive to the sensed movement of the finger of the hand to the position. For example, with the first level may require a user to move their finger through a full range of motion to a position to grasp or contact the virtual object. The graphics engine 125 may not provide any assistance, and this finger support level 155 may be a basic support or low support level. The first level may define a level of collision and effective grip with 100% finger locking and adaptability of the object in hand.

A second level of finger support 155 can cause the graphics engine 125 to animate the hand grasping the virtual object responsive to the sensed movements of the finger to a second position at least halfway between an original position of the finger and the position to grasp the virtual object. For example, with the second level of finger support 155, the patient may only need to move their finger halfway between an open hand position and the surface of the virtual object before the graphics engine 125 animates the finger moving to and touching the virtual object to grasp the virtual object. The second level of finger support 155 can be an intermediate or medium finger support level 155, with effective level of collision and grip with 50% finger closure and adaptability of the object in hand.

A third level of finger support 155 can cause the graphics engine 125 to animate the hand grasping the virtual object responsive to the sensed movement of the finger to a third position less than halfway between the original position of the finger and the position to grasp the virtual object. For example, if the finger of the patient moves a fifth of the way between an open hand position and a surface of the virtual object, the graphics engine 125 can animates the finger moving to and touching the virtual object to grasp the virtual object. The third level of finger support 155 can be an advanced support or high level of support for effective collision and grip level with 20% finger locking and adaptability of the object in hand.

For example, if the rehabilitation level 150 can detect, determine, or identify an increase in the rehabilitation level 150 of the patient, the support selector 145 can decrease the level of assistance to provide the patient to grasp the virtual object using the increase of the rehabilitation level 150 of the patient. For example, responsive to detecting an increase in the rehabilitation level 150 by a threshold amount, the support selector 145 can decrease the support or level of assistance provided to the patient. For example, if the support selector 145 identifies an increased the rehabilitation level 150 above a threshold, the support selector 145 can decrease the amount of assistance provided to the patient. With the selected level of assistance (or the decreased level of assistance), the computing system 105 can animate the hands, fingers, or arms of the patient grasping the virtual object on the XR equipment 110.

Each finger support level 155 can be associated with a different threshold or set of thresholds. For example, the high finger support level 155 may have a first threshold. The support selector 145 can compare the rehabilitation level 150 to the first threshold. If the rehabilitation level 150 is less than the first threshold, the support selector 145 can select the high finger support level. However, if the rehabilitation level 150 is greater than the first threshold, but less than a second threshold, the support selector 145 can select the medium level of finger support 155. However, if the rehabilitation level 150 is greater than the third threshold, the support selector 145 can select the basic or low level of finger support 155.

With the selected finger support level 155, the graphics engine 125 can animate the motions or movements of the fingers or hands of the patient. For example, the graphics engine 125 can cause the position of the virtual fingers to match or track the positions of the actual fingers of the patient using data received from the sensors 120. The graphics engine 125 can receive, from the XR equipment 110, sensed movement of a finger of the hand of the patient a distance towards a position to grasp a virtual object. The graphics engine 125 can compute, determine, or measure the distance traveled by the finger relative to an origin or other position. For example, the distance can be measured from a position where the use has an open palm where fingers of the patient are parallel with the surface of the palm. The distance can be measured between the origin position to a position defined based on the surface of a virtual object that the patient is attempting to grasp. The graphics engine 125 can animate, on the XR equipment 110, the finger of the hand of the patient grasping the virtual object responsive to the distance exceeding a threshold of the selected finger support level 155. For example, the threshold can be set by the support selector 145 based on the rehabilitation level 150. The threshold of the finger support level 155 can be twenty percent of the way between the origin position and the surface of the virtual object, half-way between the origin position and the surface of the virtual object, or all the way, or substantially all the way to the surface of the virtual object.

For example, the graphics engine 125 can receive data from the sensors 120. The graphics engine 125 can detect, determine or compute the sensed movement of a finger of the hand of the patient a distance to a second position short of a position to grasp the virtual object (e.g., the surface of the virtual object). The graphics engine 125 can compare the distance that the finger of the patient moved with or against the threshold of the selected finger support level 155. Responsive to the distance satisfying (e.g., being equal to or exceeding the threshold), the graphics engine 125 can animate the finger to move from the second position to the position to grasp the virtual object. For example, the graphics engine 125 can model the motion of the finger that the finger would take to bend, curl, or move to grasp the virtual object.

The support selector 145 can select different grasping posture levels 160. For example, the graphics engine 125 can animate various different hand and finger postures to grasp a virtual object. However, the support selector 145 can store indications of which postures to activate or enable based on the rehabilitation level 150 of a patient. In some implementations, the finger support levels 155 are sub-divided by the grasping postures 160. The support selector 145 can combine each finger support level 155 with a different grasping posture level 160. The grasping postures 160 can be sub-levels of the finger support levels 155. For each or a set of virtual objects in a XR environment, the support selector 145 can implement a selected grasping posture 160.

The support selector 145 can store multiple grasping posture levels 160. The support selector 145 can store one, two, three, or any number of grasping posture levels 160. A first grasping posture level 160 can include a first number of postures for grasping interactions between a hand of the patient and a virtual object. A second grasping posture level 160 can include a second number of postures for grasping interactions between a hand of the patient and a virtual object. A third grasping posture level 160 can include a third number of postures for grasping interactions between a hand of the patient and a virtual object. The second number of postures can be less than the first number of postures. The third number of postures can be less than the second number. The postures can be radial digital grasp pattern, pincer grasp, raking grasp, gross grasp pattern, power grasp, or any other type of posture or finger pattern to grasp an object. Different postures can be associated with different levels of difficulty, and therefore, basic grasping posture levels 160 can include simpler or easier grasping postures, while advanced grasping posture levels 160 can include additional advanced grasping postures.

The support selector 145 can select from various different grasping posture levels 160 using the rehabilitation level 150. For example, the support selector 145 can compare the rehabilitation level 150 to multiple thresholds. If the rehabilitation level 150 is greater than a first level, the support selector 145 can select a first or advanced grasping posture level 160. If the rehabilitation level 150 is less than the first level, but greater than a second level, the support selector 145 can select a second or intermediate grasping posture level 160. If the rehabilitation level 150 is less than the second level, the support selector 145 can select a third or basic grasping posture level 160.

The graphics engine 125 can identify a set of postures to hold a virtual object using the level of rehabilitation of the patient. For example, the graphics engine 125 or the support selector 145 can store a map between the grasping posture level 160 and the grasping postures available for the level 160. The graphics engine 125 can activate or enable the grasping postures corresponding to the selected grasping posture level 160 using the mapping. The graphics engine 125 can animate the hand or fingers of a patient to grasp a virtual object using at least one posture of the identified set of postures. For example, the graphics engine 125 can track the movements of the hand or fingers of the patient using data of the sensors 120. The graphics engine 125 can detect that the hand or fingers of the patient are moving to one of the postures of the set of postures. Responsive to detecting that the hand of fingers of the patient have moved to the posture of the set of postures, the graphics engine 125 can animate the hand of the patient grasping the virtual object according to the posture.

The graphics engine 125 can compare positions of the hand or fingers of the patient to an expected position of the hand or finger of the patient to grasp the virtual object. Responsive to the hands or fingers of the patient being within threshold distances from the position to grasp the virtual object, the graphics engine 125 can animate or move the hands or fingers to the positions to grasp the virtual object with the posture. The graphics engine 125 can lock the hands or fingers of the patients to a surface of the virtual object in the corresponding posture. The graphics engine 125 can measure or determine a distance from a point on each finger of the patient (e.g., a fingertip) and a position on a surface of the virtual object to grasp the virtual object corresponding to the posture. The graphics engine 125 can run a pattern matching algorithm, such as a machine learning algorithm, to determine whether the hand and finger of the patient are close enough to the expected position of the hand or finger of the patient to animate the hand or fingers of the patient to grasp the virtual object. The graphics engine 125 can compare the distances to thresholds, and if the distances are less than the thresholds, animate the fingers in the positions to hold or grasp the object corresponding to the posture. The graphics engine 125 can lock the virtual object to the hand of the patient so that the patient can carry or move the virtual object.

The first grasping posture level 160 can be an advanced postures for an advanced patient, e.g., a patient with a rehabilitation level 150 greater than a first threshold. The first grasping posture level 160 can include a high or highest number of different grasping posture to grasp a virtual object in order to challenge an advanced or highly rehabilitated patient. The patient can use the each of the different types of postures of the first grasping posture level 160 to grasp the virtual object. For example, the advanced grasping posture level 160 can provide ten different postures of grasping interaction that allow for ten different ways of grasping any shape of a virtual object.

The second grasping posture level 160 can be for an intermediate patient, e.g., a patient with a rehabilitation level 150 greater than a second threshold, but less than the first threshold. The intermediate grasping posture level 160 can include five postures of grasping interaction allowing ten different ways of grasping any shape of a virtual object. The third grasping posture level 160 can be for a beginner patient, e.g., a patient with a rehabilitation level 150 less than the second threshold. The intermediate grasping posture level 160 can include two postures of grasping interaction allowing ten different ways of grasping any shape of a virtual object.

The computing system 105 can include at least one exercise selector 165. The exercise selector 165 can select or transition between different therapeutic tasks or training exercises to rehabilitate a patient's neuromotor capabilities. The exercise selector 165 can store different types of tasks for a patient to perform to train the patient to move their arms, hands, or fingers to grasp, hold, and move virtual objects in the XR environment. The exercise selector 165 can store multiple, or a set of different XR environments or XR environment configurations. The exercise selector 165 can configure the positions of various targets for a patient to move a virtual object to and can configure the positions for virtual objects to be initiated at within the XR environment.

The configurations 170-185 can be based on ICF standards, movement taxonomy, and principles of motor learning. ICF establishes a standardized and universal framework for describing disability and related macro dimensions of functioning, establishing three main levels, the first component being impairments in physiological functions and anatomical structures, the second being limitations in individual activities or tasks, and the third being restrictions in participation in life situations. The functional movement taxonomy of the upper extremity characterizes fundamental movements that allow humans to perform activities of daily living, which include reaching, affordance, grasping, carrying, stabilizing, supporting, repositioning and resting. The principles of motor learning and neuroplasticity establish through research in clinical and theoretical neuroscience several mechanisms that favor the learning of new functions or relearning of lost ones. The configurations 170-185 of the exercise selector 165 can be designed based on task difficulty manipulation, visuo-motor discordance, explicit feedback, implicit feedback, effector selection, increased practice times, increased repetitions, spaced practice, variable practice, task-specific practice, goal-oriented practice, embodied practice, embodied practice, motor problem solving, multisensory stimulation, motor imagery, gamification, rhythmic cues, and social interaction.

The exercise selector 165 can include a horizontal configuration 170. The horizontal configuration 170 can define positions of targets and virtual objects. The positions of targets and virtual objects can be defined by the horizontal configuration 170 so that the patient picks up and moves the virtual objects along a horizontal axis to the targets. The horizontal configuration 170 can define various levels of elevations for the targets. The computing system 105 can determine a rehabilitation level 150 using sensed movements of the sensors 120. The exercise selector 165 can receive the rehabilitation level 150 from the computing system, e.g., from the machine learning model 190. As the patient successfully moves the virtual object to the target, and the rehabilitation level 150 increases, the exercise selector 165 can increase the elevation of the targets. For example, the exercise selector 165 can periodically or iteratively increase an elevation of the targets responsive to increases of at least a threshold in the rehabilitation level 150. For example, responsive to determining that the rehabilitation level 150 has increased by a predefined amount, the exercise selector 165 can increase the elevation of the target by a predefined amount. The computing system 105 can determine the rehabilitation level 160 based on the elevation of the targets. For example, when determining the rehabilitation level 160, the computing system 105 can place greater weight on a patient successfully moving a virtual object to a first higher target than a patient moving a virtual object to a second lower target.

The exercise selector 165 can include a vertical configuration 175. The vertical configuration 175 can define positions of targets and virtual objects. The positions of targets and virtual objects can be defined by the vertical configuration 175 so that the patient picks up and moves the virtual objects along a vertical axis to the targets. The vertical configuration 175 can define various levels of elevations for the targets.

The exercise selector 165 can include a horizontal and vertical configuration 180. The horizontal and vertical configuration 180 can define positions for targets and virtual objects. A position of a first target and a first virtual object can be defined by the configuration 180 so that the patient moves the virtual object along a horizontal axis to the first target. A position of a second target and a second virtual object can be defined by the configuration 180 so that the patient moves the second virtual object along a vertical axis to the second target.

The exercise selector 165 can include a semi-circular configuration 185. The exercise selector 165 can define positions for targets and virtual objects that are positioned about an arc or semi-circle. For example, the targets can be disposed about a first arc with a first arc length and the virtual objects can be disposed about a second arc with a second arc length less than the first arc length. The exercise selector 165 can define the positions of the virtual objects and targets so that the patient moves each virtual object along a different axis to the corresponding target.

The graphics engine 125 can animate, on the XR equipment 110, a XR environment including a virtual object and a first target for a patient to move the virtual object from a starting position along a first axis to the first target. For example, the positions of the virtual object and the target can be defined according to one of the configurations 170-185. For example, if the exercise selector 165 selects the horizontal configuration 170, the graphics engine 125 can render or animate the virtual object and the target at the positions defined by the horizontal configuration 170 so that the user moves the virtual object along a horizontal axis to the target.

The computing system 105 can receive sensed movements via the sensors 120 of the patient moving the virtual object to the target. The performance identifier 195 or the machine learning model 190 can determine the rehabilitation level 150 using the sensed motions. For example, the computing system 105 can receive data identifying the positions, motions, or movements of the fingers, hands, head, or arms of the patient grasping the virtual object and moving the virtual object to the target. The computing system 105 can record, track, or count each repetition of the patient successfully moving the virtual object to the target. The computing system 105 can determine the rehabilitation level 150 based on the number of attempts to move the virtual object to the target and the number of successful attempts of the patient to move the virtual object to the target.

As the patient completes repetitions of grasping and moving the virtual object to the target, the rehabilitation level 150 can increase. Responsive to the rehabilitation level 150 satisfying or exceeding a threshold, the exercise selector 165 can update the XR environment to change the training of the patient or increase the difficulty of the training. The computing system 105 can update, on the XR equipment 110, the XR environment to include a second target for the patient to move the virtual object along a second axis to responsive to the sensed movements of the hand of the patient indicating at least a threshold level of rehabilitation. For example, if the exercise selector 165 selects the horizontal configuration 170, the sensed motions can be the patient moving the virtual object along the horizontal axis to the target. However, once the rehabilitation level 150 increases above a threshold, the exercise selector 165 can change the position of the target or the virtual object according to the vertical configuration 175 so that the patient moves the virtual object along a vertical axis instead of the horizontal axis to the patient. Once the rehabilitation level 150 increases by another threshold amount, the exercise selector 165 can select the horizontal and vertical configuration 180 and the graphics engine 125 can implement the horizontal and vertical configuration 180. Once the rehabilitation level 150 increases by another threshold amount, the exercise selector 165 can select the semi-circular configuration 185 and the graphics engine 125 can implement the semi-circular configuration 185.

The computing system can determine rehabilitation levels 150 for each hand of the patient, e.g., a left hand rehabilitation level 150 and a right hand rehabilitation level 150. The computing system 105 can receive right hand data from the sensors 120 tracking the motions or movements of the right hand of the patient. The computing system 105 can determine the right hand rehabilitation level 150 using the right hand data. The computing system 105 can receive left hand data from the sensors 120 tracking the motions or movements of the left hand of the patient. The computing system 105 can determine the left hand rehabilitation level 150 using the left hand data. The graphics engine 125 can receive, from the XR equipment 110, sensed movements of the right hand of the patient grasping the virtual object and moving the virtual object along the first axis to the first target. The graphics engine 125 can receive, from the XR equipment 110, the sensed movements of a left hand of the patient grasping a second virtual object and moving the second virtual object along the first axis to another target. For example, the exercise selector 165 can select the horizontal configuration 170, and the computing system 105 can track the performance of the right hand and the left hand respectively of the patient moving virtual objects along a horizontal axis to respective targets. The graphics engine 125 can update, on the XR equipment 110, the XR environment to include the second target for the patient to move the virtual object along a second axis to responsive to a level of rehabilitation of the left hand of the patient meeting a threshold and a level of rehabilitation of the right hand of the patient meeting the threshold. For example, once the rehabilitation levels 150 of both the left hand and the right hand of the patient satisfy the threshold, the exercise selector 165 can select a new configuration, e.g., switch from the horizontal configuration 170 to the vertical configuration 175. For example, if the patient was moving the virtual objects along a horizontal axis, once the rehabilitation levels 150 of both hands of the patient satisfy the threshold, the graphics engine 125 can update the XR environment so that the virtual objects and targets are at positions for the patient to move the virtual objects along vertical axes to the targets.

The computing system 105 can include at least one performance identifier 195. The performance identifier 195 and the machine learning model 190 can provide a comprehensive solution to collect accurate metrics of users' functional performance, such as finger spacing or distance 197, and hand, arm, or head movement frequency or motion maps 193 using machine learning to automate and tailor rehabilitation exercises in a personalized manner.

The performance identifier 195 can determine a finger distance 197. The distance 197 can be a distance of movement of a finger of the patient. The distance 197 can be a distance that the patient is capable of moving their finger. The performance identifier 195 can determine a maximum or average distance that each finger of the patient is able to travel (on one or both hands of a patient). The performance identifier 195 can determine a finger distance 197 for each finger of each hand of a patient. For example, the performance identifier 195 can determine a maximum or average distance that each finger can bend between an open palm position and a closed fist position. The performance identifier 195 can receive motion data from the sensors 120 of the XR equipment 110, and use the received data to determine the finger distance 197.

The performance identifier 195 can determine a head position 187 using data received from the sensors 120 of the XR equipment 110. The performance identifier 195 can receive data from the sensors 120 that indicates the position or motion of the head of the patient. The head position 187 can be one or a set of head positions of the patient. The head position 187 can indicate an average position of a head of the patient. The head position 187 can indicate a distance that a patient can bend or move their head. The head position 187 can track the position or positions of the head of the patient using the data received from the XR equipment 110.

The performance identifier 195 can determine posture function 183. The performance identifier 195 can receive data from the XR equipment 110 indicating the motions or movements of the fingers and the hands of the patient. The performance identifier 195 can track the patient's attempts to form different postures with their hands, e.g., opposition, curl, abduction, fist, flat, etc. The performance identifier 195 can track the positions or motions of the fingers or hands of the patient and determine how closely the position of the fingers of the hand of the patient match a particular posture. The closer the patient can move their fingers into a particular posture, the higher the posture functionality for the posture. The performance identifier 195 can determine an indicator or score for each posture, e.g., opposition, curl, abduction, fist, flat, etc. The score for each posture can indicate an ability of the user to make a particular hand posture.

The performance identifier 195 can generate or update a frequency or heat map 193 using sensed movements of a portion of the patient received from the sensors 120 of the XR equipment 110. The heat map 193 can represent movements of a hand, arm, finger, or head of a patient in the XR environment. The heat map 193 can be a two-dimensional or three- dimensional map. The heat map 193 can identify movements of a portion of a patient in the XR environment at multiple different positions. For example, the performance identifier 195 can identify the frequency or length of time that a portion of the patient is in different locations within the XR environment. For example, the map 193 can be a two-dimensional map that includes an x axis and a y axis (e.g., left and right and forward and back axes). The two- dimensional heat map 193 can be a continuous set of positions or discrete positions. The map 193 can be a three-dimensional map that includes x, y, and z axis (e.g., left and right, forward and back, and up and down axes). For example, the map 193 can be a volumetric representation of motions of a center of a hand of a user. The map 193 can be a two-dimensional or three-dimensional matrix or tensor. The dimensions of the matrix can correspond to the dimensions of the environment displayed by the graphics engine 125 or the dimensions of a work area (such as a table) where the patient performs neuromotor rehabilitation. This can cause the map 193 to be anchored to the environment such that the map 193 can be viewed consistently by the patient via the XR equipment 110 from a variety of different angles or positions within the environment. Each entry of the matrix can correspond to a small square shaped portion, pixel, or block of the environment displayed by the graphics engine 125. At least one position of the map 193 can be associated or store with an indication or value quantifying the length of time that a hand was at the position.

The performance identifier 195 can iteratively update the heat map 193. For example, as new data is received from the sensors 120, or at an interval, the performance identifier 195 can update the heat maps 193. The performance identifier 195 can determine, based on a sensed motion of a hand of a patient received from the sensors 120, a position of a hand of the patient in the environment and a length of time that the hand was present at the position. Based on the length of time that the hand was in the position, the performance identifier 195 can increase a value in the map 193 associated with the position. In this regard, the values of the map 193 can be iteratively or procedurally updated or increased overtime. Responsive to a detection that a hand of a patient has not been at a particular point for at least a threshold length of time, the performance identifier 195 can decrease a value in the map 193 associated with the particular point.

Performance identifier 195 can generate heat maps 193 for individual hands of a patient. For example, the performance identifier 195 can receive sensed movements of the right hand of a patient and sensed movements of the left hand of the patient via the sensors 120 of the XR equipment 110. The performance identifier 195 can track the motions, movements, or positions of the right hand of the patient and update a right-hand heat map 193 using the motions. The performance identifier 195 can track the motions, movements, or positions of the left hand of the patient and update a left-hand heat map 193 using the motions.

The computing system 105 can include at least one machine learning model 190. The machine learning model 190 can be a neural network, such as a convolutional neural network. The convolutional neural network can be a 3D convolutional neural network that includes three-dimensional filters used by the model to perform convolutions. The machine learning model 190 can be a 2D or 3D one-pass multi-task network, a U-Net, or any other image processing or classification model. The computing system 105 can train a model 190 with supervised, unsupervised, or self-supervised training techniques. The computing system 105 can train a model 190 with a training dataset to output the rehabilitation level 150. The computing system 105 can receive the training data from an external database or data source. The computing system can receive rehabilitation levels 150 from a user device to tag different heat maps 193 collected over time. The computing system 105 can store training data that includes maps 193 and corresponding rehabilitation levels 150. For example, each two-dimensional or three- dimensional map of the training data can be tagged or associated with a rehabilitation level 150. The training data can include historical finger distance 197, posture function 183, or head position 187. The computing system 105 can train the model 190 to output a rehabilitation level 150 for a given heat map 193 input to the model 190. Once the model 190 is trained and has an accuracy greater than a threshold, the computing system 105 can deploy the model 190. The computing system 105 can deploy the model 190 by setting the model to execute or activate the model 190 to execute. If the model 190 is trained on a remote computing system, deploying the model 190 can include transmitting the model to the computing system 105 or the XR equipment 110.

The machine learning model 190 can determine rehabilitation levels 150 for the left hand or right hand of a patient. The computing system 105 can execute the machine learning model 190 on a heat map 193 of positions of a left hand of a patient to determine a left hand rehabilitation level 150. The computing system 105 can execute the machine learning model 190 on a heat map 193 of positions of a right hand of a patient to determine a right hand rehabilitation level 150. The computing system 105 can compare the left hand heat map 193 with the right hand heat map 193 to identify which hand the patient favors over the other. For example, the model 190 can execute on both maps 193 together to compare the maps against each other. The computing system 105 can execute a comparison algorithm to compare the heat maps 193 and identify which hand the patient favors. The computing system 105 can compare the left hand rehabilitation level 150 with the right hand rehabilitation level 150 to identify which hand the patient favors. The computing system 105 can identify that the patient favors the right hand over the left hand if the right hand rehabilitation level 150 is greater than (greater by at least a threshold amount) the left hand rehabilitation level 150. The computing system 105 can identify that the patient favors the left hand over the right hand if the left hand rehabilitation level 150 is greater than (greater by at least a threshold amount) the right hand rehabilitation level 150.

The machine learning model 190 or the performance identifier 195 can determine the rehabilitation level 150 of the patient by executing with one or multiple heat maps 193. In some implementations, the computing system 105 can run multiple different machine learning models 190 on different heat maps 193 and combine the outputs of the different models to determine the rehabilitation level 150. For example, the machine learning model 190 can run on an individual heat map 193 to determine the rehabilitation level 150. For example, the machine learning model 190 can determine the rehabilitation level 150 from an individual heat map 193 of a hand of a patient or a head of a patient. In some implementations, one or more machine learning models 190 can determine the rehabilitation level 150 from a combination of heat maps 193, e.g., from a heat map 193 of a hand of a patient and a heat map 193 of a head of a patient. In this regard, the computing system 105 can take into account both hand movements and head movements of the patient in determining the rehabilitation level 150.

The exercise selector 165 can generate or select a set of tasks or a training exercise for the patient to perform responsive to a detection that the patient favors one hand over another. For example, the exercise selector 165 can determine that the patient should perform more exercises with their left hand than with their right hand because the patient favors their right hand, and the left hand requires additional rehabilitation. The exercise selector 165 can cause the graphics engine 125 to display the targets or objects for the patient to use to perform the tasks.

The computing system 105 can train a machine learning model 190 for each training exercise that the graphics engine 125 animates for the patient. For example, because each exercise selected by the exercise selector 165 can require the patient to move their hands or fingers to different positions, the computing system 105 can train a machine learning model 190 for each training exercise. The computing system 105 can generate a heat map 193 for each individual training exercise, and select and apply the model 190 for the corresponding training exercise to the heat map 193. The computing system 105 can combine multiple rehabilitation levels 150 output by different models 190 for different training exercises to track a composite indication of the patients level of rehabilitation.

The computing system 105 can execute the model 190 to generate an inference. The computing system 105 can execute the model 190 with collected performance identifier 195 to determine the rehabilitation level 150. For example, the computing system 105 can execute the model 190 with a two-dimensional or three-dimensional heat map 193 to determine the rehabilitation level 150. The computing system 105 can execute the model 190 with the head position 187, the finger distance 197, or the posture function 183 to output the rehabilitation level 150.

The computing system 105 can use the rehabilitation level 150 to change the difficulty of the training exercises presented to the patient on the XR equipment 110. For example, the computing system 105 can provide the rehabilitation level 150 output by the machine learning model 190 to the exercise selector 165. The exercise selector 165 can compare the rehabilitation level 150 to a threshold. Based on the comparison, the exercise selector 165 can update tasks to be performed by increasing a level of difficulty of the tasks that the user completes or the training for the patient. For example, if the exercise selector 165 currently has the vertical configuration 175 selected for animating training exercises for the user, the exercise selector 165 can switch to the horizontal/vertical configuration 180 responsive to the rehabilitation level 150 increasing above a threshold level or increasing by a threshold amount. For example, if the exercise selector 165 currently has the horizontal/vertical configuration 180 selected for animating training exercises for the user, the exercise selector 165 can switch to the semi-circular configuration 185 responsive to the rehabilitation level 150 increasing above a threshold level or increasing by a threshold amount.

The graphics engine 125 can switch between VR and MR. For example, a user can provide an input via the XR equipment 110, e.g., via a button press or gesture, indicating to switch between VR and MR. Responsive to receiving the user input, the graphics engine 125 can toggle between rendering an entirely virtual environment or a fully immersive virtual environment for display on the display 115 of the XR equipment 110 and a partially virtual environment. In the fully virtual environment mode, the graphics engine 125 can render a surrounding of the patient, an avatar of the patient (e.g., body, arms, hands, etc.), and virtual objects for the patient to move. In the fully virtual environment mode, the graphics engine 125 may make an environment of the patient fully immersive, and nothing the patient sees with the XR equipment 110 may be real. However, in the MR environment, a surrounding environment of the patient can be viewed by the patient (e.g., either directly or as a camera feed captured by camera of the XR equipment 110), but overlaid with virtual elements, e.g., the virtual objects. In this regard, when doing exercises, a patient can view their actual physical hand in their actual environment interacting with virtual objects, e.g., picking up and moving the virtual objects. However, if desired, the patient can toggle back to viewing a virtual avatar hand interacting with the same virtual objects. In this regard, the graphics engine 125 can hide or display an avatar of the patient responsive to a user input. The finger support 155 can used by the graphics engine 125 in either the VR mode or the MR mode.

The performance identifier 195 can determine advanced kinematic metrics based on data or kinematics data collected from the XR equipment 110. The data can be data captured by motion sensors, depth cameras, and/or VR systems of the XR equipment 110. For example, using data collected from the XR equipment 110, the performance identifier 195 can determine a range of motion (ROM), such as angular amplitude of the joints of a patient in different planes, speed and acceleration of movement of limbs of a patient to evaluate fluidity and coordination, symmetry and compensation (e.g., comparisons between sides of the body or abnormal patterns in gait or movement), trajectory and movement patterns (e.g., 3D tracking to detect motor improvements or deficits), fatigue or movement variability (e.g., to evaluate the patient's endurance and stability over time), and/or 3D volumetric digital biomarkers.

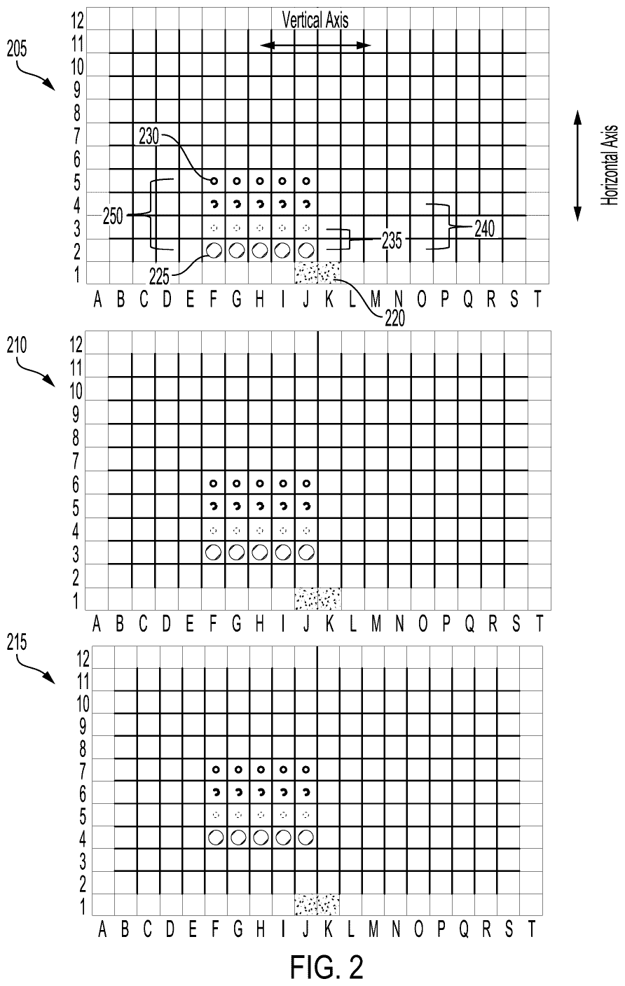

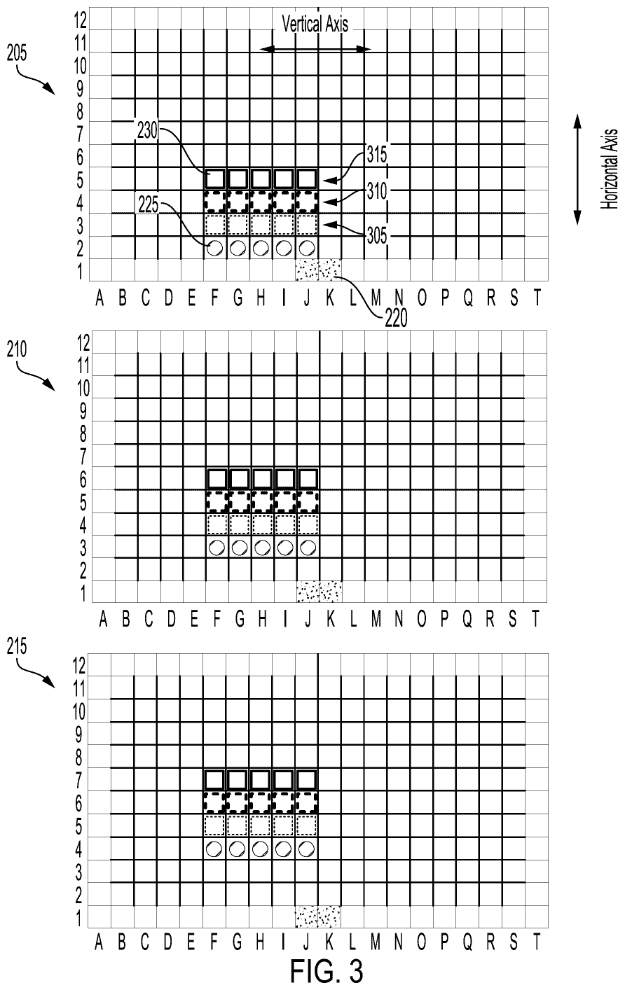

Referring now to FIGS. 2-3, among others, layouts 205-215 of a XR environment for a patient to move an object along a horizontal axis to a target are shown. The layouts 205-215 can represent the positions of virtual objects in a virtual training space or area. The virtual training area can be or include a table. The virtual training area can be a rectangular space. Each layout 205-215 can identify the positions for virtual objects 225 and targets 230. The virtual objects 225 can be three dimensional shapes, such as cubes, prisms, pyramids, or spheres. The virtual objects 225 can be everyday objects such as cups, plates, utensils, etc. The targets 230 can be marks or locations where the patient is to move the virtual objects 225. The targets 230 can be bullseye targets, spheres, squares, x-shaped marks, etc. The targets 230 can be containers for a patient to place the virtual object 225 onto or inside of, such as a vase, a cup, a plate.

The exercise selector 165 can select a layout 205, 210, or 215, and render or animate the XR environment according to the selected layout. For example, the graphics engine 125 can animate, on the XR equipment 110, targets 230 at different distances from a position 220 of the patient for the patient to move virtual objects 225 to. The virtual objects 225 and the targets 230 can be disposed along an axis parallel to a horizontal axis, such that when the patient picks up and moves the virtual objects 225 to the targets, the patient is moving the virtual objects 225 away from the position 220 in the same direction as the horizontal axis.

The graphics engine 125 can receive, from the XR equipment 110, sensed movements of the hand of the patient grasping the virtual objects 225 and moving the virtual objects to the targets 230. The environment can include multiple targets 230 that are different distances from a starting position of a virtual object 225. For each virtual object 225, the layouts 205-215 can include a first target a first distance from the virtual object 225, a second target a second distance from the virtual object 225 greater than the first distance, and a third target a third distance from the virtual object 225 greater than the second distance. The graphics engine 125 can determine, using sensed movements via the sensor 120 and the different distances, the rehabilitation level 150 of the patient. The farther the patient is able to move the virtual object 225, the more rehabilitated the patient may be. The performance identifier 195 can identify which targets the patient succeeds in moving the virtual objects 225 to. The performance identifier 195 can determine the rehabilitation level 150 based on the distance between targets 230 and the starting point of the virtual object 225. The performance identifier 195 can determine the rehabilitation level 150 to be higher if the patient moves the virtual object farther.

The graphics engine 125 can first animate the XR environment according to the layout 205. For example, the graphics engine 125 can render the virtual objects 225 at positions indicated by the layout 205 and render the targets 230 at positions indicated by the layout 205. The graphics engine 125 can update the XR environment according to the layout 210 responsive to the rehabilitation level 150 of the patient increasing to at least a threshold level. The graphics engine 125 can render the virtual objects at positions indicated by the layout 210 and render the targets 230 at positions indicated by the layout 210. The graphics engine 125 can update the XR environment according to the layout 215 responsive to the rehabilitation level 150 of the patient increasing to at least a threshold level. The graphics engine 125 can render the virtual objects 225 at positions indicated by the layout 210 and render the targets 230 at positions indicated by the layout 215. As the patient progresses through the layouts 205-215, the virtual objects 225 and targets 230 can move farther and farther away from the position 220 of the patient along a horizontal axis to challenge the patient and effectively rehabilitate the patient.

A first row of targets 230 can have a distance 235 in a horizontal direction from each respective virtual object 225. The first distance 235 can be 9-11 centimeter. The first distance 235 can be 8-12 centimeter. The first distance 235 can be less than 8 centimeters. The first distance 235 can be more than 12 centimeters. A second row of targets 230 can have a distance 240 in a horizontal direction from each respective virtual object 225. The second distance 240 can be 19-21 centimeters. The second distance 240 can be 18-22 centimeters. The second distance 240 can be less than 18 centimeters. The second distance 240 can be more than 22 centimeters. A third row of targets 230 can have a distance 250 in a vertical direction from each respective virtual object 225. The third distance 250 can be 29-31 centimeters. The third distance 250 can be 28-32 centimeters. The third distance 250 can be less than 28 centimeters. The third distance 250 can be more than 32 centimeters.

In FIG. 3, different elevations 305-315 for rows of targets are shown for the layouts 205-215. A first row of targets 230 can have a first elevation 305. The first elevation 305 can be 9-11 centimeters. The first elevation 305 can be 8-12 centimeters. The first elevation 305 can be less than 8 centimeters. The first elevation 305 can be more than 12 centimeters. The second row of targets 230 can have a second elevation 310. The second elevation 310 can be 19-21 centimeters. The second elevation 310 can be 18-22 centimeters. The second elevation 310 can be less than 18 centimeters. The second elevation 310 can be more than 22 centimeters. A third row of targets 230 can have a third elevation 315. The third elevation 315 can be 29-31 centimeters. The third elevation 315 can be 28-32 centimeters. The third elevation 315 can be less than 28 centimeters. The third elevation 315 can be more than 32 centimeters.

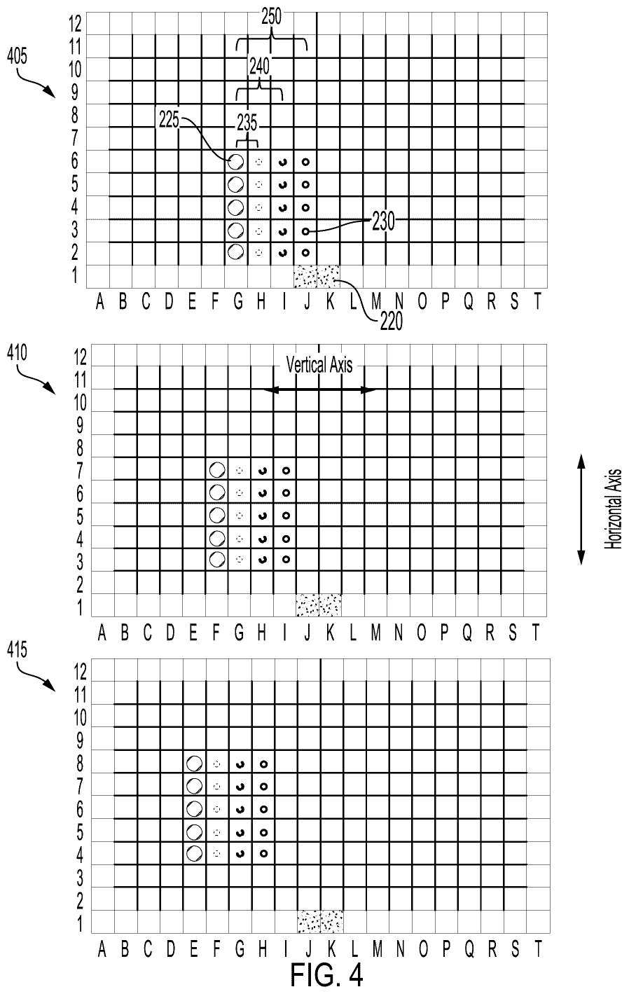

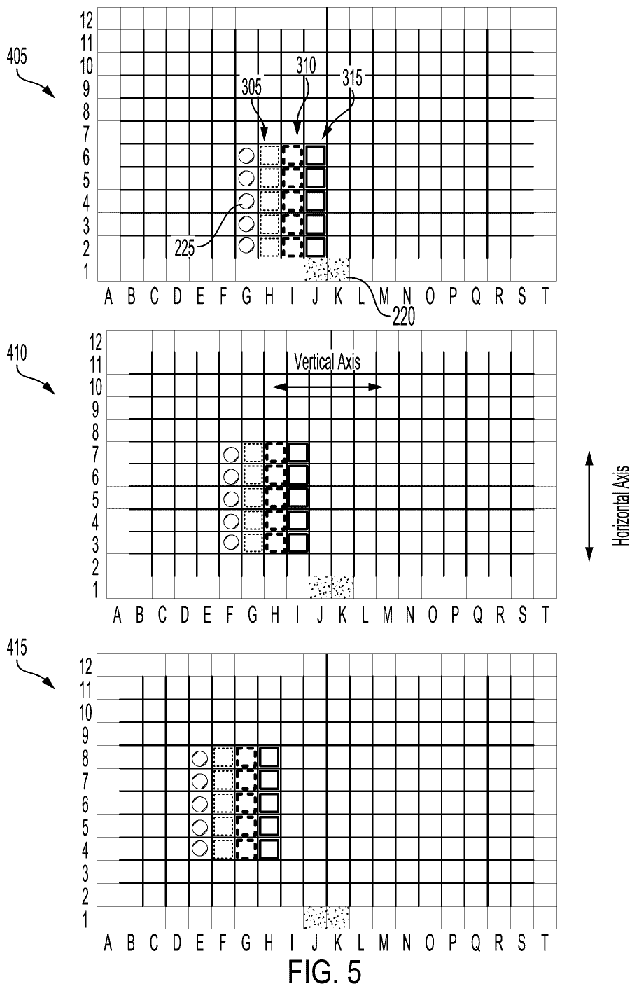

Referring now to FIGS. 4-5, among others, layouts 405-415 of a XR environment for a patient to move an object along a vertical axis to a target are shown. The layout 405 can define the positions for the virtual objects 225 and the targets 230. The positions of the virtual objects 225 and the targets 230 can be set so that the user moves the virtual objects 225 along a vertical axis to the targets 230. The positions can be set so that the user moves the virtual objects 225 from a starting position back towards the position 220 of the patient along a vertical axis. FIG. 5 illustrates different elevations 305, 310, and 315 for respective columns of the targets.

The graphics engine 125 can first animate the XR environment according to the layout 405. For example, the graphics engine 125 can render the virtual objects at positions indicated by the layout 405 and render the targets 230 at positions indicated by the layout 405. The graphics engine 125 can update the XR environment according to the layout 410 responsive to the rehabilitation level 150 of the patient increasing to at least a threshold level. The graphics engine 125 can render the virtual objects at positions indicated by the layout 410 and render the targets 230 at positions indicated by the layout 410. The graphics engine 125 can update the XR environment according to the layout 415 responsive to the rehabilitation level 150 of the patient increasing to at least a threshold level. The graphics engine 125 can render the virtual objects at positions indicated by the layout 415 and render the targets 230 at positions indicated by the layout 415. As the patient progresses through the layouts 405-415, the virtual objects 225 and targets 230 can move farther and farther away from the position 220 of the patient along a vertical axis to challenge the patient and effectively rehabilitate the patient.

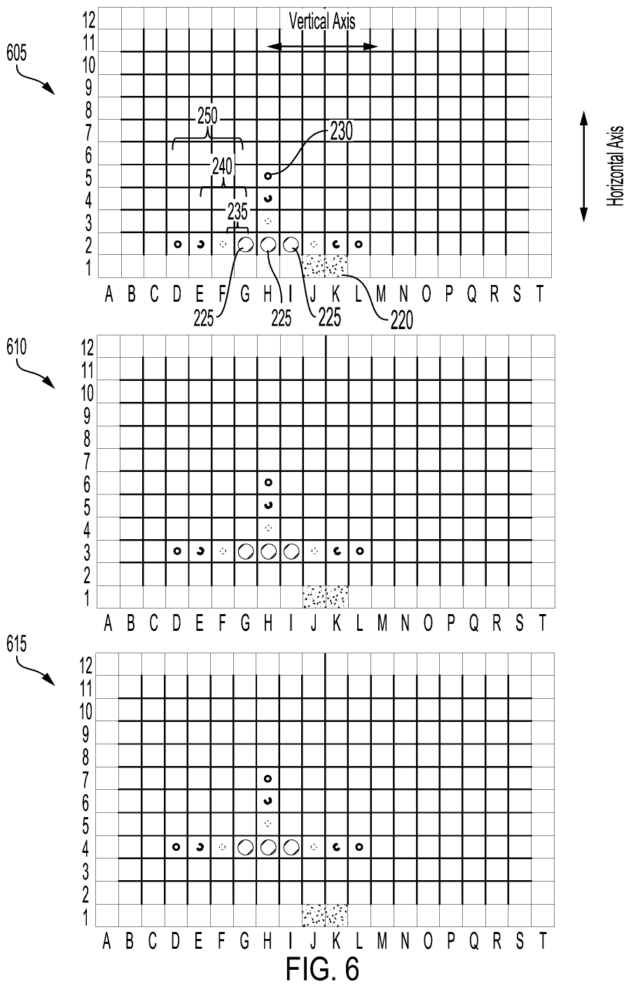

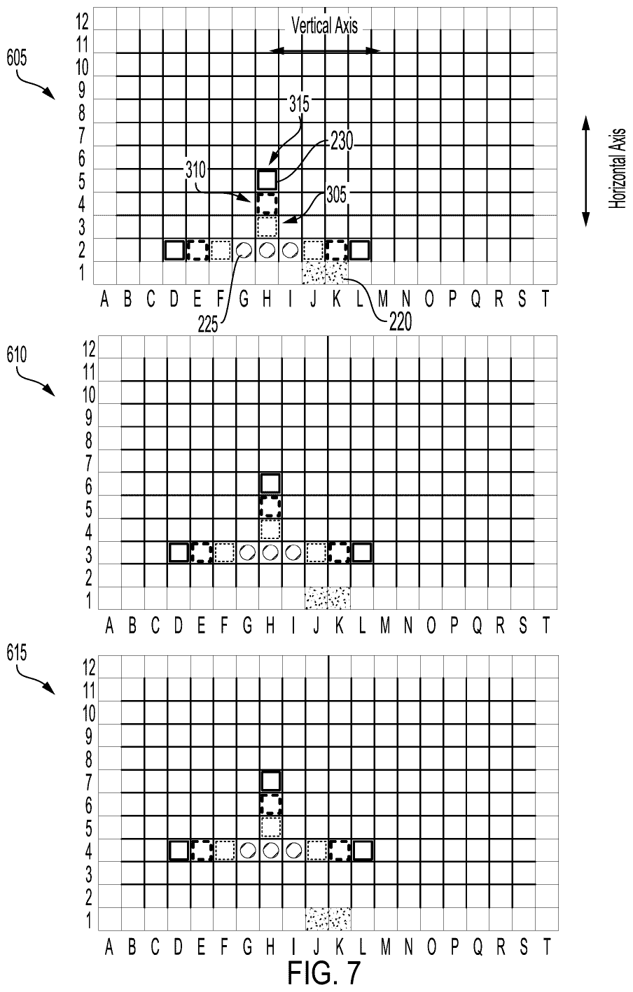

Referring now to FIGS. 6-7, among others, layouts 605-615 of a XR environment for a patient to move objects along a vertical axis and a horizontal axis to targets are shown. A first virtual object 225 can be positioned according to the layouts 605-615 such that the patient moves the virtual object along a vertical axis away from a position 220 of a patient to targets 230. A second virtual object 225 can be positioned according to the layouts 605-615 such that the patient moves the virtual object along a vertical axis towards a position 220 of a patient to targets 230. A third virtual object 225 can be positioned according to the layouts 605-615 such that the patient moves the virtual object along a horizontal axis away from a position 220 of a patient to targets 230. The targets 230 can have different elevations 305-315. The targets 230 can increase in elevation along a direction that the patient moves the virtual object 225.

The targets 230 and virtual objects 225 can be moved away from a position 220 of a patient along a vertical axis. For example, layout 605 can position the targets 230 and the virtual objects 225 closest to a position 220 of the patient. The layout 610 can move the targets 230 and the virtual objects 225 a first distance away from the position 220 of the patient along the vertical axis. The layout 615 can move the targets 230 and the virtual objects 225 a second distance greater than the first distance away from the position 220 of the patient along the vertical axis. The exercise selector 165 can begin with the layout 605, and switch to the layout 610 responsive to detecting an increase in the rehabilitation level 150 by a threshold amount, and switch to the layout 615 responsive to another increase in the rehabilitation level 150 by a threshold amount.

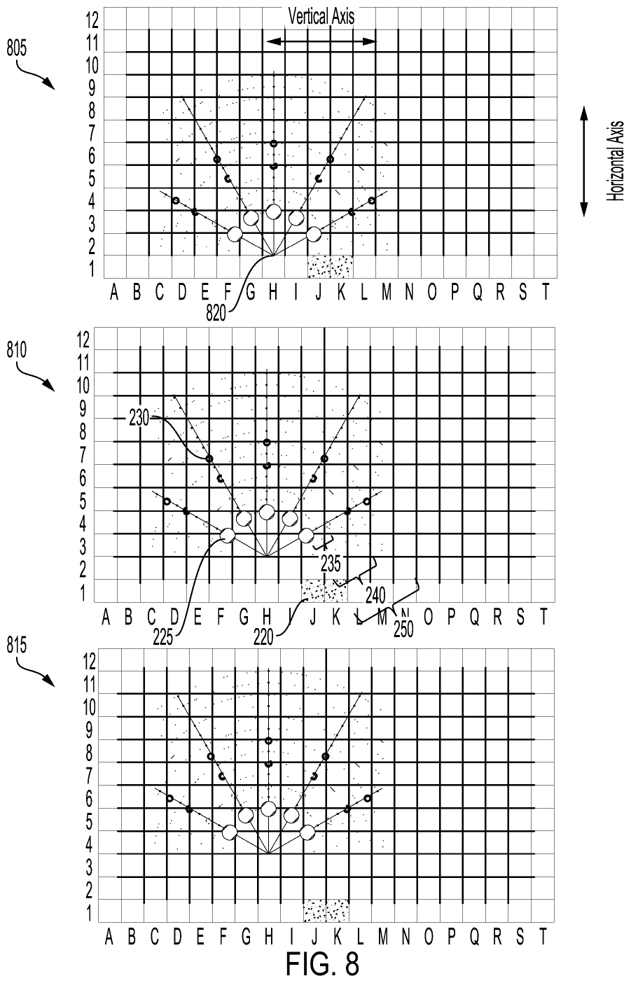

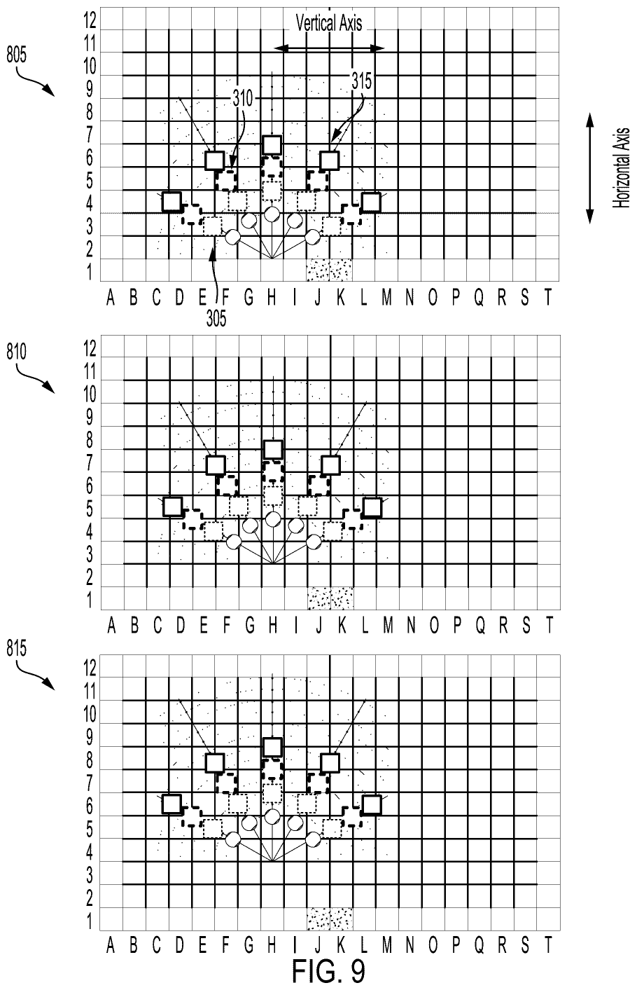

Referring now to FIGS. 8-9, among others, layouts 805-815 of a XR environment for a patient to move virtual objects 225 spaced in a semi-circle along axes to targets 230 are shown. The targets 230 can be positioned in a semi-circle around a position 820 and virtual objects 225 for the patient to move from starting positions along respective axes to the targets 230. The virtual objects 225 and the targets 230 can be positioned on semi-circles or arcs of increasing radius. For each virtual object 225, a set of targets 230 can be positioned in a line or respective axis with a set of virtual object 225. The patient can move each virtual object 225 along the respective axis to the respective targets 230. The performance identifier 195 can receive data from the sensors 120 of the patient grasping the virtual objects 225 and moving the virtual objects 225 along the respective axes to the targets 230. The performance identifier 195 can use the sensed movements of the patient grasping and moving the virtual objects 225 to determine or update the rehabilitation level 150.

The targets 230 can have different elevations 305-315. The targets 230 can increase in elevation along a direction that the patient moves the virtual object 225. Furthermore, the virtual objects 225, the targets 230, and the position 820 can move forward away from the position 220 of the patient along a vertical axis as rehabilitation level 150 of the patient improves. The exercise selector 165 can begin with using the layout 805. Responsive to the rehabilitation level 150 of the patient increasing by a threshold amount or increasing to a threshold level, the exercise selector 165 can update the XR environment with the layout 810. Responsive to the rehabilitation level 150 of the patient increasing by a threshold amount or increasing to a threshold level, the exercise selector 165 can update the XR environment with the layout 815.

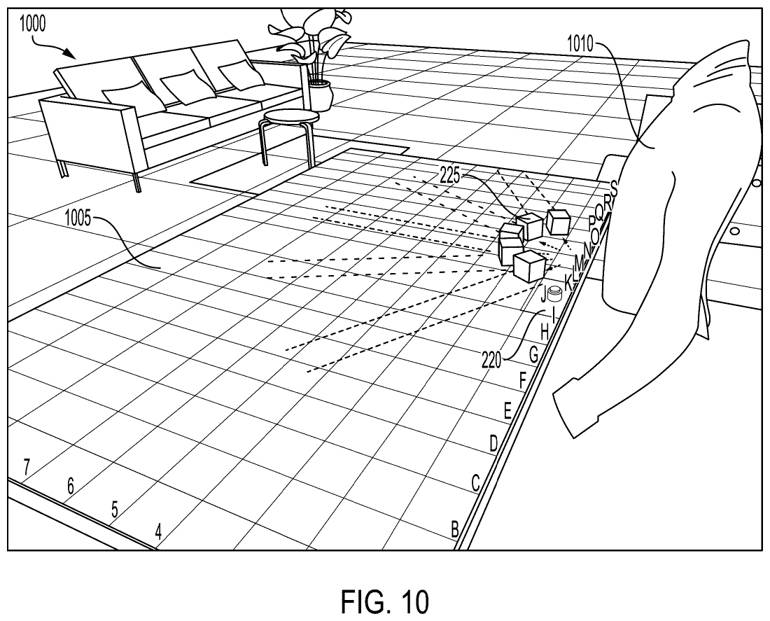

Referring now to FIG. 10, among others, an example XR environment 1000 for a patient to move virtual objects 225 spaced in a semi-circle along axes to targets is shown. In environment 1000, an avatar 1010 identifying the position and orientation of the patient is included. Furthermore, the layout 805 is selected and implemented to configure the positions of the virtual objects 225 on a table 1005. The surface 1005 can be any flat surface that is elevated.

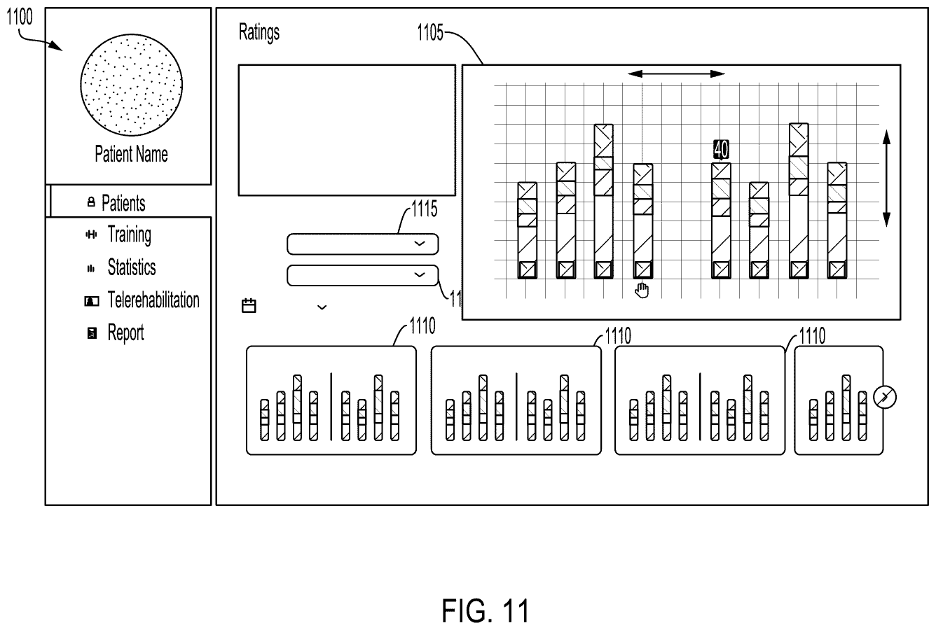

Referring now to FIG. 11, among others, depicts an electronic display 1000 including two-dimensional heat maps depicting performance of a patient moving virtual objects along a horizontal axis to targets is shown. The electronic display 1100 can be generated by the computing system 105, and displayed on an electronic display of a client device, such as a laptop computer, a desktop computer, a tablet, a smartphone, a console, etc. The electronic display 1100 can be displayed on the display 115 of the XR equipment 110 as an element or component of a XR environment.

The electronic display 1100 can include a selectable element 1115 that a user can use to select an exercise. The electronic display 1100 can include a selectable element 1120 that a user can use to switch between viewing data and measurements for a left hand and a right hand of a patient. The electronic display 1100 can include an element 1105 indicating heat map data for a particular exercise. For example, the element 1105 can depict a heat map 193 for exercises or training moving virtual objects to targets positioned according to the layout 205. The heat map 193 can include a column or band that corresponds to the direction that the patient moves the virtual object. The element 1105 can depict a heat map 193 for a left hand of the patient and a heat map 193 for a right hand of the patient. Different measurements 1110 can be displayed in the electronic display 1100, and can be selected by a user.

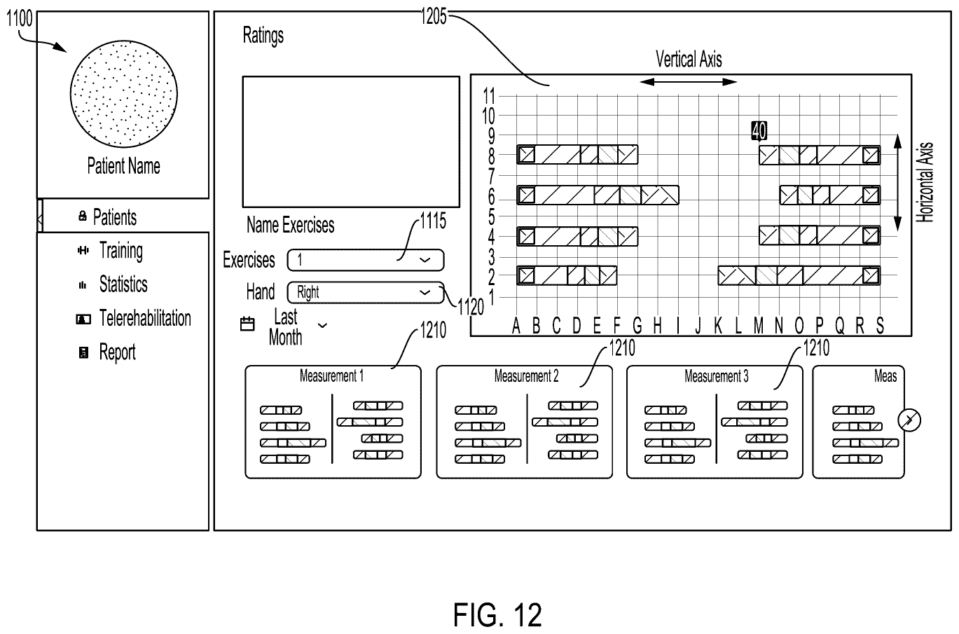

Referring now to FIG. 12, among others, an electronic display 1100 including two- dimensional heat maps depicting performance of a patient moving virtual objects along a vertical axis to a target is shown. The electronic display 1100 can include an element 1205 indicating heat map data for a particular exercise. For example, the element 1205 can depict a heat map 193 for exercises or training moving virtual objects to targets positioned according to the layout 405. The heat map 193 can include a row or band that corresponds to the direction that the patient moves the virtual object. The element 1205 can depict a heat map 193 for a left hand of the patient and a heat map 193 for a right hand of the patient. Different measurements 1210 for different training sessions can be displayed in the electronic display 1100, and can be selected by a user for viewing.

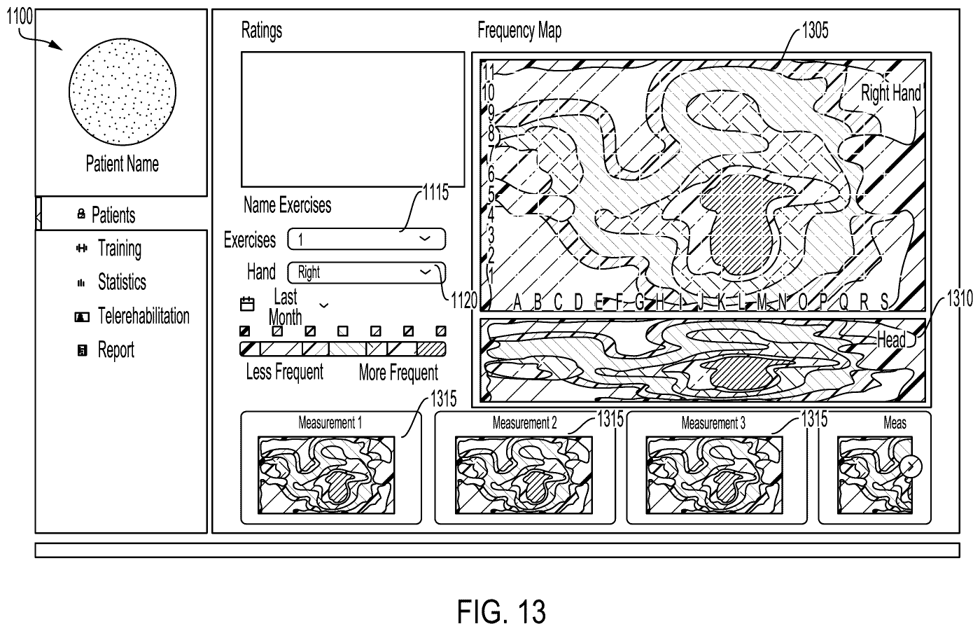

Referring now to FIG. 13, among others, an electronic display 1100 comprising a three-dimensional heat map depicting positions of a hand of a patient is shown. The electronic display 1100 can include at least one overhead element 1305. The overhead element 1305 can indicate a heat map 193 for a XR environment from an overhead perspective. The overhead element 1305 can include a heat map 193 for a left hand or a right hand, based on the selection in the selectable element 1120. The element 1305 can include two lateral axes, e.g., an x-axis and a y-axis. The underlying heat map 193 rendered in the element 1305 can be a three dimensional heatmap. The overhead element 1305 can indicate the heat map 193 in two dimensions. The computing system 105 can reduce the three dimensional heat map 193 to a two dimensional heat map 193 before displaying the heat map 193 in the element 1305. For example, the computing system 105 can sum, average, or combine together each value in the heatmap for a particular x-coordinate and y-coordinate for an entire range of elevations (e.g., over an entire range of z-coordinates), and save the resulting value in a two dimensional heatmap at the corresponding x-coordinate and y-coordinate. The electronic display 1100 can include an element 1310 to display a heatmap for a head of a patient. The element 1310 can indicate a heatmap in two dimensions, e.g., a lateral axis and an elevation axis, indicating the positions of a head of a patient. The electronic display 1100 can include measurements 1315 of different heat maps 193 at different times that a user can select and view.

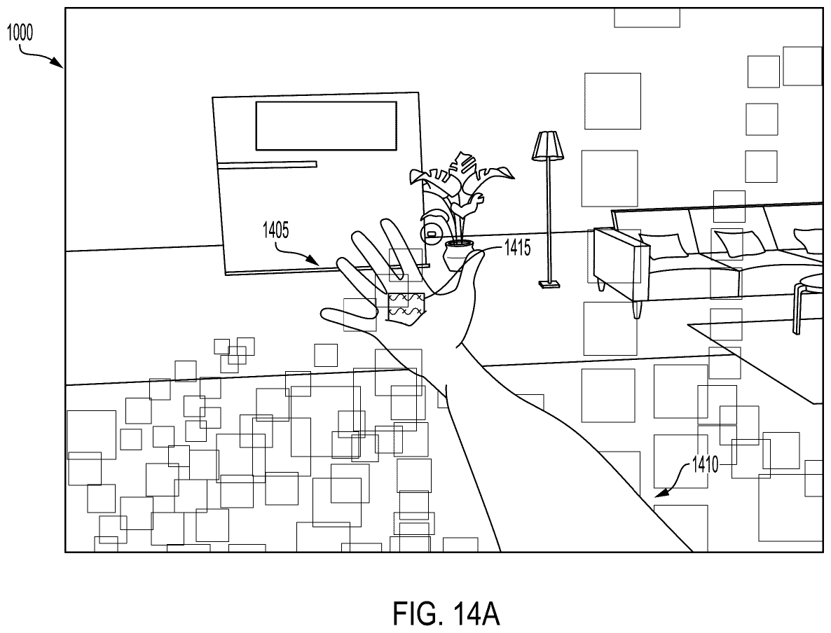

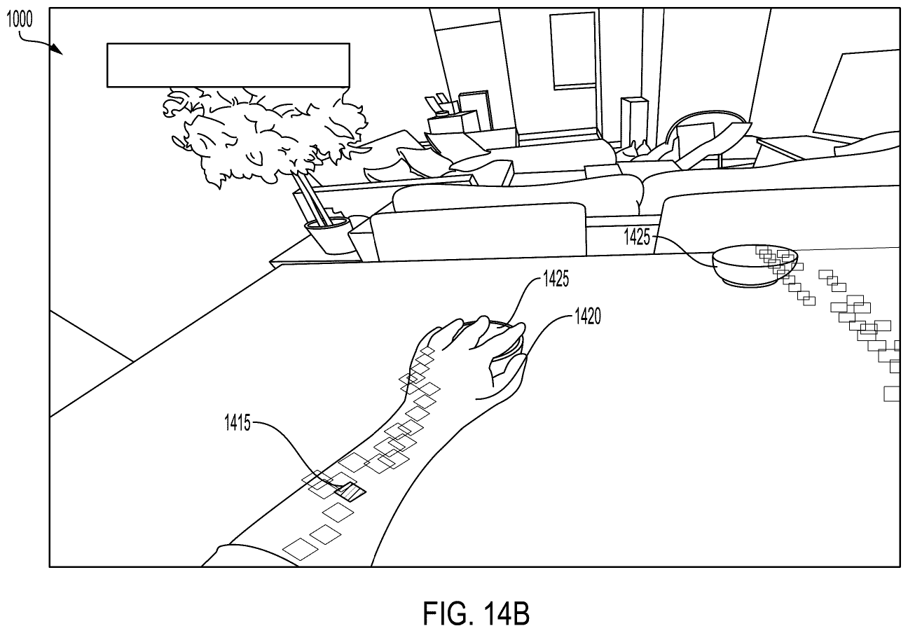

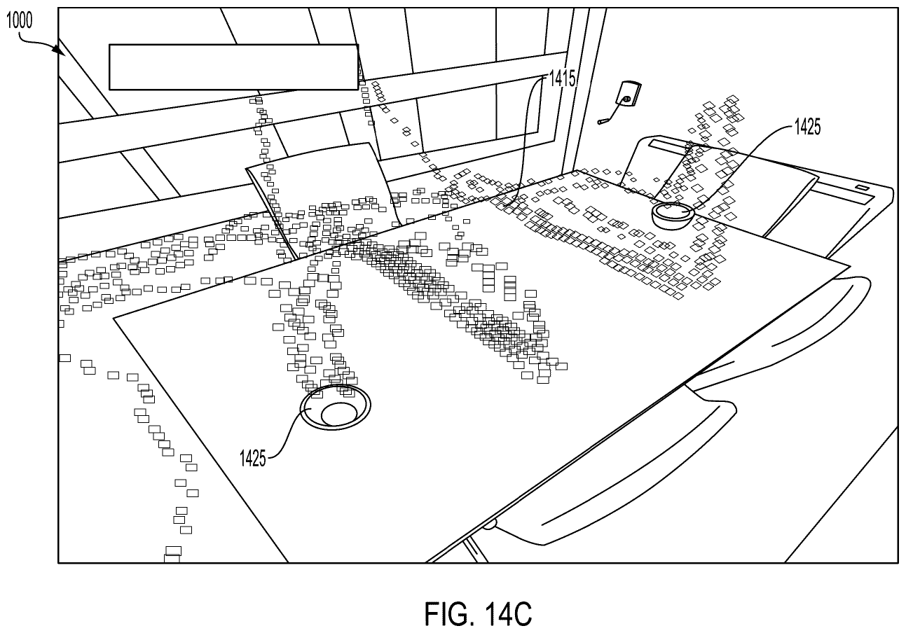

Referring now to FIG. 14A, among others, a XR environment 1000 tracking a three-dimensional heat map depicting positions of a hand and arm of a patient is shown. The XR environment 1000 can include a virtual hand 1405 and a virtual arm 1410 that are part of the avatar 1010 of the patient. The virtual hand 1405 or arm 1410 can be displayed with an orientation, posture, and position corresponding to the sensed movements of the hands and arms of the patient. The XR environment 1000 can include indications 1415 that indicate the frequency that the hand of the patient was in a particular position within the XR environment 1000. The indicators 1415 can be shapes, cubes, colors, or regions of the XR environment 1000. The computing system 105 can, for a given position, only display an indicator 1415 responsive to a value of the indicator 1415 exceeding a threshold. For example, the computing system 1100 may only display an indicator 1415 for a position within the XR environment 1000 if the patient has put their hand in the position for at least a threshold length of time.

Referring now to FIGS. 14B-14C, a XR environment 1000 tracking a three-dimensional heat map 193 depicting positions of a hand of a patient is shown. In FIGS. 14B-14C, the XR environment 1000 can be a MR environment. The XR environment 1000 can be an MR environment such as a real-world view overlaid with virtual graphics and information rendered by the graphics engine 125 and displayed by the XR equipment 110. The XR environment 1000 can be overlaid with the heat map 193. For example, in FIGS. 14B and 14C, a user can see their own limbs (e.g., arms, hands, etc.). The user can move their limbs to move physical objects 1425 (or alternatively virtual objects 225). The XR equipment 110 can track the motions of the hands 1420 (e.g., track a center of the hands 1420) of the user via sensors 120, e.g., via a camera video feed. The performance identifier 195 can generate the heat map 193 based on the motions of the hands of the user and determine values or levels for each point, block, pixel, 3D shape, region, or indicator 1415 indicating how long the hand of the user was in each point in the XR environment 1000. The graphics engine 125 can render each indicator 1415 of the heat map 193 as a different color (or color intensity) to indicate what positions the hand 1420 has been in. For example, the longer a center of a hand 1420 of a user remained in a particular position, the more intense the color of the indicator 1415 corresponding to the particular position can be rendered by the graphics engine 125.

In FIG. 14C, a user wearing the XR equipment 110 can view the heat map 193 from the side. For example, the heat map 193 can be generated or recorded while the user is seated at the table moving the objects 1425. However, the heat map 193 can be generated to include coordinates corresponding to the XR environment 1000. A link, map, or relationship can be used to translate the coordinates of the heat map 193 into the coordinates of the XR environment 1000. In this regard, when the user gets up from the table, and views the XR environment 1000 from a different position within the XR environment 1000, the heat map 193 can remain anchored in the environment such that the user can accurately view the positions their hand 1420 was in from a variety of angles or positions.

The performance identifier 195 can determine or generate a motion speed value or indicator for the hand 1420 of the user. For example, the performance identifier 195 can use the heat map 193 to determine how rapidly the hand 1420 of the user was moving (e.g., look at a difference in indicators 1415). For example, the lower the length of time the user dwells in a particular position indicated by the indicator 1415, the faster the hand 1420 of the user is moving. The performance identifier 195 can feed the speed values into the machine learning model 190 to determine a rehabilitation level 150.

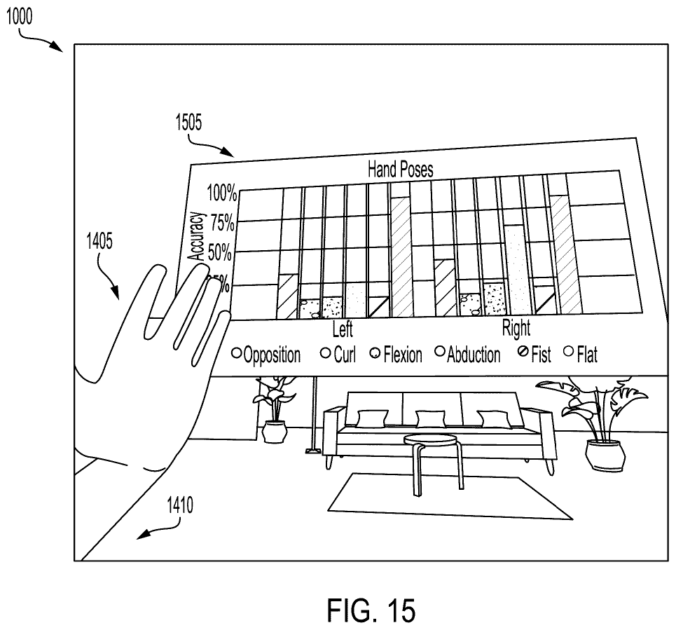

Referring now to FIG. 15, among others, a XR environment 1000 including performance indicators of hand poses is shown. The XR environment 1000 can include a chart 1505. The chart 1505 can plot a patient's ability to put their left and right hands in different hand poses, e.g., opposition, curl, flexion, abduction, fist, flat, etc. The graphics engine 125 can render the chart 1505 within the XR environment 1000 responsive to a request, responsive to a patient completing a training level or exercise, or responsive the patient's ability to make one or more hand poses increased by a threshold level. The graphics engine 125 can lock the chart 1505 to a position within the XR environment 1000, such that when the patient moves about the XR environment 1000, the chart 1505 does not move. The graphics engine 125 can lock the chart 1505 to a field of view of the patient, such that the patient views the chart 1505 on the display 115 regardless of the position or orientation of the patient in the XR environment 1000.

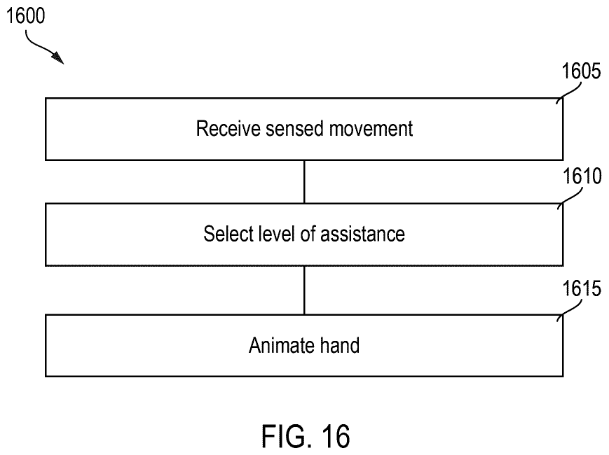

Referring now to FIG. 16, among others, an example method 1600 of selecting a level of assistance to provide a patient to grasp a virtual object is shown. The computing system 105 can perform at least a portion of the method 1600. The XR equipment 110 can perform at least a portion of the method 1600. Any kind of computing system, computing environment, server, controller, processing circuit, or processor can perform at least a portion of the method 1600. The method 1600 can include at least one ACT 1605 of receiving sensed movement. The method 1600 can include at least one ACT 1610 of selecting a level of assistance. The method 1600 can include an ACT 1615 of animating a hand.

At ACT 1605, the method 1600 can include receiving, by the computing system 105, sensed movement. The method 1600 can include receiving, by the computing system 105, sensed movements from the XR equipment 110. The method 1600 can include receiving, by the computing system 105, sensed movements of the patient wearing the XR equipment 110. The method 1600 can include receiving, by the computing system 105, sensed movements of a patient via the sensors 120 of the XR equipment 110. The method 1600 can include receiving, by the computing system 105, sensed movements of a head, of hands, of arms, or of fingers of the patient. The method 1600 can include receiving, by the computing system 105, changes in position, indications of velocity, or indications of acceleration from the sensors 120.

At ACT 1610, the method 1600 can include selecting, by the computing system 105, a level of assistance. The method 1600 can include selecting, by the computing system 105, a finger support level 155. The method 1600 can include selecting, by the computing system 105, from different levels of finger support 155 using the rehabilitation level 150. For example, the more advanced the patient is, the lower the level of finger support 155 the computing system 105 can select. The method 1600 can include selecting between different finger support levels 155 that are associated with movement thresholds. The graphics engine 125 can animate fingers of a hand of an avatar of a patient closing or grasping a virtual object responsive to detections that the fingers of the patient have moved by at least the movement threshold. The method 1600 can select a finger support level 155 with a low threshold for a patient with a low level of rehabilitation 150. The method 1600 can include selecting, by the computing system 105, a finger support level 155 with a higher threshold for a patient with a higher level of rehabilitation 150.

The method 1600 can include selecting, by the computing system 105, grasping postures 160. The method 1600 can select different levels of grasping postures 160 based on the rehabilitation level 150. The method 1600 can select different hand postures to snap or fix a hand of a patient to a virtual object with based on a selected set of grasping postures 160. The method 1600 can select a larger group of grasping postures 160 for a patient with a higher level 150 of rehabilitation. The method 1600 can include selecting, by the computing system 105, a smaller group of grasping postures 160 for a patient with a lower level 150 of rehabilitation.

At ACT 1615, the method 1600 can include animating, by the computing system 105, a hand. The method 1600 can include animating, rendering, drawing, or displaying a virtual representation of a hand of a patient on the XR equipment 110. The method 1600 can include animating a virtual representation of a hand of a patient to mirror or map to the position or orientation of a physical hand of a patient. For example, the method 1600 can include animating the virtual hand of the avatar of the patient using sensed movements from the sensors 120.

The method 1600 can include animating, by the computing system 105, the hand of the patient using the selected level of assistance at ACT 1610. For example, the method 1600 can include comparing a sensed movement of a finger of the patient via data from the sensors 120 to thresholds of finger support 155, to determine whether to animate the hand moving to touch or contact a virtual object. For example, if the computing system 105 determines that a finger of a patient has moved at least a threshold distance to a surface of the virtual object, the computing system 105 can animate the finger moving the rest of the way to the surface of the virtual object to assist the patient. Similarly, the method 1600 can include animating the hands or fingers of the patient to different postures based on the selected grasping postures 160. For example, the method 1600 can include comparing sensed finger positions of a hand of a patient to finger positions of different grasping postures 160, and animate the fingers of the patient to a closest matching grasping posture 160.

The method 1600 can include executing a graphics engine 125 to animate the fingers and hands of a patient with the selected assistance. The method 1600 can include executing a collider tool 130 and a XR tool 135 to animate the hand of the patient with various levels of assistance. The method 1600 can include executing an integration script 140 that integrates the collider tool 130 with the XR tool 135 to animate the hand of the patient colliding and snapping to different virtual objects.

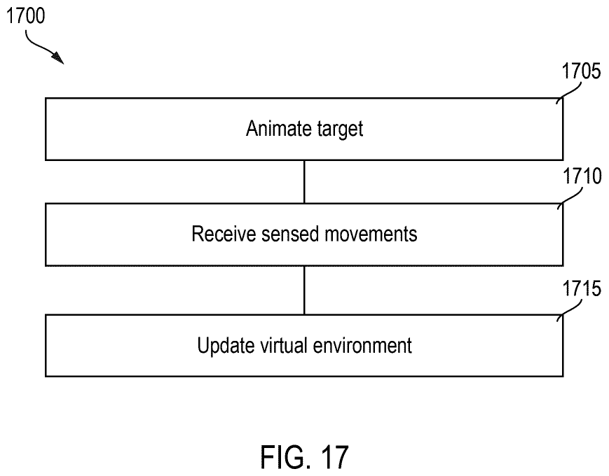

Referring now to FIG. 17, among others, an example method 1700 of updating targets of a XR environment based on a level of rehabilitation of a patient is shown. The computing system 105 can perform at least a portion of the method 1700. The XR equipment 110 can perform at least a portion of the method 1700. Any kind of computing system, computing environment, server, controller, processing circuit, or processor can perform at least a portion of the method 1700. The method 1700 can include at least one ACT 1705 of animating a target. The method 1700 can include at least one ACT 1710 of receiving sensed movements. The method 1700 can include an ACT 1715 of updating a XR environment.

At ACT 1705, the method 1700 can include animating, by the computing system 105, a target. The method 1700 can include animating or rendering a XR environment on the XR equipment 110. The method 1700 can include animating a virtual object and a target in the XR environment. The method 1700 can include animating the virtual object and target according to a configuration. For example, the method 1700 can select one of configurations 170-185 according to the rehabilitation level 150. The configuration 170-185 can define different positions of the virtual objects and targets to challenge the patient to move the virtual objects along different axes, e.g., along horizontal axis, vertical axis, vertical and horizontal axis, or axes of a semi-circular pattern. For example, the method 1700 can include animating, by the computing system 105, the virtual object and the target in positions within the virtual object so that the user moves the virtual object along a first axis to the target (e.g., along a horizontal axis).

At ACT 1710, the method 1700 can include receiving, by the computing system 105, sensed movements. The method 1700 can include receiving, by the computing system 105, sensed movements of the fingers, hands, or arms of the patient via the sensors 120. The method 1700 can include receiving sensed movements that indicate that the user has (or has attempted to) pick up the virtual object and move the virtual object to the target along (or in the same direction as) the first axis. The method 1700 can include determining, using the sensed movements, a rehabilitation level 150 of the patient. For example, the method 1700 can include determining a heat map 193 using the sensed movements, determining head position 187, finger distance 197, or posture function 183 from the sensed movements. The method 1700 can determining, by the computing system 105, the rehabilitation level 150 from the heat map 193 using the sensed movements, determining head position 187, finger distance 197, or posture function 183 from the sensed movements. The method 1700 can determine the rehabilitation level 150 from a difficulty of the movements required to move the virtual object to the target (e.g., which configuration 170-185 is being implemented by the graphics engine 125), a distance that the patient moved the virtual object, a number of repetitions of moving the virtual object to the target, numbers of successful and unsuccessful attempts at moving the virtual object to the target, elevations of the targets, etc.

At ACT 1715, the method 1700 can include updating, by the computing system 105, a XR environment. The method 1700 can include comparing the rehabilitation level 150 to a threshold. For example, each configuration 170-185 can include or be associated with a different threshold. The method 1700 can include updating, by the computing system 105, the XR environment responsive to a detection that the rehabilitation level 150 satisfies a threshold. For example, the method 1700 can include switching, by the computing system 105, from the horizontal configuration 170 to the vertical configuration 175 responsive to the rehabilitation level 150 exceeding a threshold of the vertical configuration 175.

The method 1700 can include updating the positions of the virtual objects or targets in the XR environment using the rehabilitation level 150. For example, responsive to the rehabilitation level 150 exceeding the threshold of the vertical configuration 175, the method 1700 can include updating the positions of the virtual objects or targets according to the vertical configuration 175. With the updated positions, the patient can grasp the virtual objects and move the virtual objects along the vertical axis to the targets.

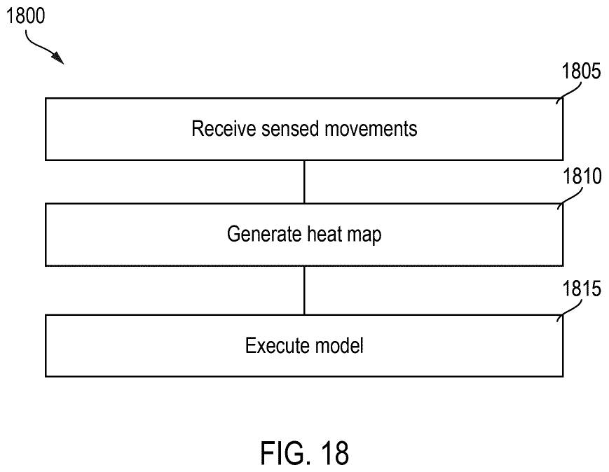

Referring now to FIG. 18, among others, an example method 1800 of generating a heat map of a XR environment depicting positions of a hand of a patient is shown. The computing system 105 can perform at least a portion of the method 1800. The XR equipment 110 can perform at least a portion of the method 1800. Any kind of computing system, computing environment, server, controller, processing circuit, or processor can perform at least a portion of the method 1800. The method 1800 can include at least one ACT 1805 of receiving sensed movements. The method 1800 can include at least one ACT 1810 of generating a heat map. The method 1800 can include an ACT 1815 of executing a model.

At ACT 1805, the method 1800 can include receiving, by the computing system 105, sensed movements. The method 1800 can include receiving, by the computing system 105, linear accelerations or rotational accelerations from the sensors 120 that track the accelerations of the arms, hands, or fingers of the patient to track the position of the arms, hands, or fingers of the patient in the XR environment. The method 1800 can include tracking, by the computing system 105, a coordinate indicating the position of the hand of the patient in the XR environment. The method 1800 can include recording, by the computing system 105, dwell times indicating a length of time that the hand of the patient was at each coordinate.

At ACT 1810, the method 1800 can include generating, by the computing system 105, a heat map 193. The method 1800 can include generating, by the computing system 105, the heat map 193. The method 1800 can include generating, by the computing system 105, the heat map 193 using the sensed movements. The method 1800 can include generating a heat map 193 to be or include at least one two or three dimensional matrix. The method 1800 can include updating the matrix as sensed movements of the hand of the patient are collected. The method 1800 can include adjusting a value of the matrix using a dwell time corresponding to a coordinate that the hand of the patient was positioned at.

At ACT 1815, the method 1800 can include executing, by the computing system 105. The method 1800 can include inputting, by the computing system 105, the heat map 193 to the model 190. The method 1800 can include executing, by the computing system 105, the model 190 with the heat map 193 to output the rehabilitation level 150. The method 1800 can include executing, by the computing system 105, the model 190 with other additional or alternative inputs, such as head position 187, finger distance 197, posture function 183, a heat map of head positions, etc. The method 1800 can selecting, by the computing system, between different models to execute. For example, the computing system 105 can store a model 190 for each configuration 170-185. Each model can be trained with data collected from each respective configuration. The method 1800 can include identifying, by the computing system 105, the configuration currently implemented or for which data was collected. The method 1800 can include retrieving, by the computing system 105, the model 190 corresponding to the identified configuration. The method 1800 can include executing, by the computing system 105, the retrieved model 190 using the heat map 193 to output the rehabilitation level 150.

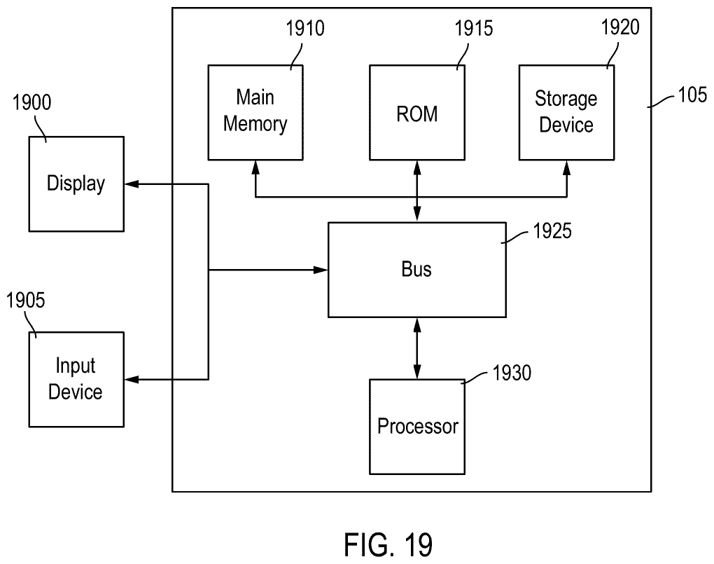

Referring now to FIG. 19, among others, an example block diagram of a computing system 105 is shown. The computing system 105 can include or be used to implement a data processing system or its components. The architecture described in FIG. 19 can be used to implement the computing system 105 or the XR equipment 110. The computing system 105 can include at least one bus 1925 or other communication component for communicating information and at least one processor 1930 or processing circuit coupled to the bus 1925 for processing information. The computing system 105 can include one or more processors 1930 or processing circuits coupled to the bus 1925 for processing information. The computing system 105 can include at least one main memory 1910, such as a random access memory (RAM) or other dynamic storage device, coupled to the bus 1925 for storing information, and instructions to be executed by the processor 1930. The main memory 1910 can be used for storing information during execution of instructions by the processor 1930. The computing system 105 can further include at least one read only memory (ROM) 1915 or other static storage device coupled to the bus 1925 for storing static information and instructions for the processor 1930. A storage device 1920, such as a solid state device, magnetic disk or optical disk, can be coupled to the bus 1925 to persistently store information and instructions.

The computing system 105 can be coupled via the bus 1925 to a display 1900, such as a liquid crystal display, or active matrix display. The display 1900 can display information to a user. An input device 1905, such as a keyboard or voice interface can be coupled to the bus 1925 for communicating information and commands to the processor 1930. The input device 1905 can include a touch screen of the display 1900. The input device 1905 can include a cursor control, such as a mouse, a trackball, or cursor direction keys, for communicating direction information and command selections to the processor 1930 and for controlling cursor movement on the display 1900.

The processes, systems and methods described herein can be implemented by the computing system 105 in response to the processor 1930 executing an arrangement of instructions contained in main memory 1910. Such instructions can be read into main memory 1910 from another computer-readable medium, such as the storage device 1920. Execution of the arrangement of instructions contained in main memory 1910 causes the computing system 105 to perform the illustrative processes described herein. One or more processors in a multi-processing arrangement can be employed to execute the instructions contained in main memory 1910. Hard-wired circuitry can be used in place of or in combination with software instructions together with the systems and methods described herein. Systems and methods described herein are not limited to any specific combination of hardware circuitry and software.

Although an example computing system has been described in FIG. 19, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C #, PROLOG, Python, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices including cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. ACTs, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any ACT or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

1-40. (canceled)

41. A system, comprising:
one or more processors, coupled with memory, to:
receive, from extended reality equipment, sensed movements of a portion of a patient attempting to move a virtual object in a computer rendered environment displayed on the extended reality equipment;
generate, using the sensed movements, a three-dimensional frequency heat map indicating movements of the portion of the patient in the computer rendered environment; and
execute a model trained by machine learning using the three-dimensional frequency heat map to determine a level of rehabilitation of the patient.

42. The system of claim 41, comprising:
the one or more processors to:
execute the model to determine the level of rehabilitation of the patient based on a frequency heat map of a hand of a patient or a frequency heat map of a head of the patient individually; or
execute the model to determine the level of rehabilitation of the patient based on a combination of the frequency heat map of the hand of the patient or the frequency heat map of the head of the patient.

43. The system of claim 41, comprising:
the one or more processors to:
animate, on the extended reality equipment, a task to be completed by the patient;
compare the level of rehabilitation of the patient to a threshold; and
update, on the extended reality equipment, the task to increase a level of difficultly of the task responsive to a determination that the level of rehabilitation of the patient satisfies the threshold.

44. The system of claim 41, comprising:
the one or more processors to:
receive a training data set comprising a plurality of three-dimensional frequency heat maps tagged with a plurality of levels of rehabilitation of patients;
execute at least one machine learning technique to train the model using the training data set; and
deploy the model to determine the level of rehabilitation of the patient using the three- dimensional frequency heat map.

45. The system of claim 41, comprising:
the one or more processors to:
generate data to cause a graphical user interface to display on a user device;
the graphical user interface comprising a two-dimensional chart comprising two lateral axes to display an overhead view of the three-dimensional frequency heat map.

46. The system of claim 41, comprising:
the one or more processors to:
receive, from the extended reality equipment, first sensed movements of a right hand of the patient in the computer rendered environment and second sensed movements of a left hand of the patient in the computer rendered environment;
generate, using the first sensed movements, the three-dimensional frequency heat map indicating positions of the right hand in the computer rendered environment;
generate, using the second sensed movements, a second three-dimensional frequency heat map indicating positions of the left hand in the computer rendered environment;
execute the model trained by machine learning using the three-dimensional frequency heat map to determine the level of rehabilitation of the right hand of the patient; and
execute the model trained by machine learning using the second three-dimensional frequency heat map to determine a second level of rehabilitation of the left hand of the patient.

47. The system of claim 41, comprising:
the one or more processors to:
receive, from the extended reality equipment, first sensed movements of a right hand of the patient in the computer rendered environment and second sensed movements of a left hand of the patient in the computer rendered environment;
generate, using the first sensed movements, the three-dimensional frequency heat map indicating positions of the right hand in the computer rendered environment;
generate, using the second sensed movements, a second three-dimensional frequency heat map indicating positions of the left hand in the computer rendered environment;
compare the three-dimensional frequency heat map with the second three-dimensional frequency heat map to detect that the patient favors the right hand over the left hand; and
generate a set of tasks for the patient to complete in the computer rendered environment with the left hand responsive to a detection that the patient favors the right hand over the left hand.

48. The system of claim 41, comprising:
the one or more processors to:
receive, from the extended reality equipment, data indicating a position of a head of the patient;
track positions of the head of the patient using the data received from the extended reality equipment; and
determine the level of rehabilitation of the patient using the positions of the head of the patient.

49. The system of claim 41, comprising;
the one or more processors to:
receive, from the extended reality equipment, data indicating a distance of movement of a finger of the patient; and
determine, the level of rehabilitation of the patient using the distance of movement of the finger of the patient.

50. The system of claim 41, comprising:
the one or more processors to:
receive, from the extended reality equipment, data indicating movements of fingers of the patient; and
determine, using the data, a plurality of levels indicating an ability of the patient to make a plurality of hand poses.

51. A method, comprising:
receiving, by one or more processors, from extended reality equipment, sensed movements of a portion of a patient attempting to move a virtual object in a computer rendered environment displayed on the extended reality equipment;
generating, by the one or more processors, using the sensed movements, a three- dimensional frequency heat map indicating movements of the portion of the patient in the computer rendered environment; and
executing, by the one or more processors, a model trained by machine learning using the three-dimensional frequency heat map to determine a level of rehabilitation of the patient.

52. The method of claim 51, comprising:
animating, by the one or more processors, on the extended reality equipment, a task to be completed by the patient;
comparing, by the one or more processors, the level of rehabilitation of the patient to a threshold; and
updating, by the one or more processors, on the extended reality equipment, the task to increase a level of difficultly of the task responsive to a determination that the level of rehabilitation of the patient satisfies the threshold.

53. The method of claim 51, comprising:
receiving, by the one or more processors, a training data set comprising a plurality of three-dimensional frequency heat maps tagged with a plurality of levels of rehabilitation of patients;
executing, by the one or more processors, at least one machine learning technique to train the model using the training data set; and
deploying, by the one or more processors, the model to determine the level of rehabilitation of the patient using the three-dimensional frequency heat map.

54. The method of claim 51, comprising:
generating, by the one or more processors, data to cause a graphical user interface to display on a user device;
the graphical user interface comprising a two-dimensional chart comprising two lateral axes to display an overhead view of the three-dimensional frequency heat map.

55. The method of claim 51, comprising:
receiving, by the one or more processors, from the extended reality equipment, first sensed movements of a right hand of the patient in the computer rendered environment and second sensed movements of a left hand of the patient in the computer rendered environment;
generating, by the one or more processors, using the first sensed movements, the three- dimensional frequency heat map indicating positions of the right hand in the computer rendered environment;
generating, by the one or more processors, using the second sensed movements, a second three-dimensional frequency heat map indicating positions of the left hand in the computer rendered environment;
executing, by the one or more processors, the model trained by machine learning using the three-dimensional frequency heat map to determine the level of rehabilitation of the right hand of the patient; and
executing, by the one or more processors, the model trained by machine learning using the second three-dimensional frequency heat map to determine a second level of rehabilitation of the left hand of the patient.

56. The method of claim 51, comprising:
receiving, by the one or more processors, from the extended reality equipment, first sensed movements of a right hand of the patient in the computer rendered environment and second sensed movements of a left hand of the patient in the computer rendered environment;
generating, by the one or more processors, using the first sensed movements, the three-dimensional frequency heat map indicating positions of the right hand in the computer rendered environment;
generating, by the one or more processors, using the second sensed movements, a second three-dimensional frequency heat map indicating positions of the left hand in the computer rendered environment;
comparing, by the one or more processors, the three-dimensional frequency heat map with the second three-dimensional frequency heat map to detect that the patient favors the right hand over the left hand; and
generating, by the one or more processors, a set of tasks for the patient to complete in the computer rendered environment with the left hand responsive to a detection that the patient favors the right hand over the left hand.

57. The method of claim 51, comprising:
receiving, by the one or more processors, from the extended reality equipment, data indicating a position of a head of the patient;
tracking, by the one or more processors, positions of the head of the patient using the data received from the extended reality equipment; and
determining, by the one or more processors, the level of rehabilitation of the patient using the positions of the head of the patient.

58. The method of claim 51, comprising;
receiving, by the one or more processors, from the extended reality equipment, data indicating a distance of movement of a finger of the patient; and
determining, by the one or more processors, the level of rehabilitation of the patient using the distance of movement of the finger of the patient.

59. The method of claim 51, comprising:
receiving, by the one or more processors, from the extended reality equipment, data indicating movements of fingers of the patient; and
determining, by the one or more processors, using the data, a plurality of levels indicating an ability of the patient to make a plurality of hand poses.

60. One or more non-transitory computer readable media storing instructions thereon, that, when executed by one or more processors, cause the one or more processors to perform operations, comprising:
receiving from extended reality equipment, sensed movements of a portion of a patient attempting to move a virtual object in a computer rendered environment displayed on the extended reality equipment;
generating using the sensed movements, a three-dimensional frequency heat map indicating movements of the portion of the patient in the computer rendered environment; and
executing a model trained by machine learning using the three-dimensional frequency heat map to determine a level of rehabilitation of the patient.

61. (canceled)
